(19) United States
(12) Patent Application Publication
(10) Pub. No.: US 2025/0270381 A1
(43) Pub. Date: Aug. 28, 2025

(54) RECYCLING OF POLYESTER

(71) Applicant: Samsara Eco Pty Limited, New South Wales (AU)

(72) Inventors: Colin Jackson, St Kingston (AU); Vanessa Vongsouthi, St Gungahlin (AU); Matthew Arthur Spence, Kaleen (AU); Jeremy Nugent, St Dickson Canberra (AU); Rosemary Lee Georgelin, St Hughes (AU)

(21) Appl. No.: 18/687,550

(22) PCT Filed: Aug. 26, 2022
(86) PCT No.: PCT/AU2022/051056

§ 371 Date: Feb. 28, 2024

(30) Foreign Application Priority Data
Sep. 6, 2021 (AU) 2021902882

(51) Int. Cl. C08J 11/10; C07C 67/02; C07C 67/03; C08G 63/88; C08J 11/16; C08J 11/24

(52) U.S. Cl. CPC C08J 11/105; C07C 67/02; C07C 67/03; C08G 63/88; C08J 11/16; C08J 11/24; C08J 2367/06

(57) ABSTRACT

The present invention provides a method for recycling polyethylene terephthalate (PET), the method comprising: (i) subjecting the PET to base-catalysed transesterification with a C₆-C₁₀ mono-alcohol to produce a composition comprising one or both of a mono-ester and di-ester terephthalate C₆-C₁₀mono-alcohol derivatives; and (ii) converting the so formed one or both of mono-ester and di-ester terephthalate C₆-C₁₀mono-alcohol derivatives into terephthalic acid using an esterase.

### FIELD OF THE INVENTION

The present invention relates in general to recycling of polyester. More particularly, the invention relates to a method for recycling polyethylene terephthalate (PET).

### BACKGROUND OF THE INVENTION

The management of waste plastic products continues to be a significant problem for modern day society. Some modelling predicts as much as 90 million metric tonnes of plastic waste could end up being discarded annually into the environment by 2030 if current global trends of poor waste management continue.

It is now well documented that waste plastic is causing numerous deleterious effects to the environment.

PET is by far the most abundant polyester used in modern day society and accounts for more than 70 million metric tonnes of annual plastic production globally.

The physical and chemical properties of PET that make it so highly desirable for the use in, for example, packaging applications unfortunately also render the polymer resistant to biodegradation. Accordingly, discarded PET will persist in the environment and remain a problem for many generations to come.

Most PET-based products are produced from virgin (i.e. non-recycled) resin and ultimately end up in landfill or aquatic/marine environments. Such poor recycling management is in part caused by the inefficiency of current PET recycling technologies.

Being a thermoplastic material, PET products can technically be melt processed (for example via melt extrusion) and repurposed into recycled PET or rPET products.

While rPET certainly has a number of useful applications, recycling PET via melt processing techniques inherently promotes a reduction in the polymers physical and chemical properties. For example, the molecular weight of the PET progressively reduces the more times it undergoes melt processing resulting in a reduction in the polymers intrinsic viscosity (IV). Reduction in the IV of the so formed rPET can limit applications for rPET. Also, recycling of PET by melt processing often promotes a yellowing discolouration in the polymer, which again can limit applications for the so formed rPET.

Techniques have been developed to counter such IV reduction and yellowing discolouration in melt processed rPET. However, their effectiveness can sometimes be limited and the required additional processing requirements can add cost to the overall recycling process.

Also, melt processing in of itself demands a high energy consumption, again adding further cost to the overall recycling process

So-called chemical recycling is an alternative pathway for recycling PET. Unlike the aforementioned melt processing recycling techniques, chemical recycling actively seeks to depolymerise the PET back to the monomeric building blocks from which it is made, namely terephthalic acid (TPA) and ethylene glycol (EG) or derivatives thereof. The so formed repurposed TPA and EG can then be used as monomers in a conventional polycondensation process to produce what in effect will be a virgin PET. That virgin PET can then be used once again to produce consumer products. An advantage of chemical recycling is the resulting PET produced from the repurposed monomers will for the most part be essentially indistinguishable from virgin PET produced from monomers derived from petrochemical resources. Furthermore, chemical recycling can be undertaken indefinitely without compromising the chemical or physical properties of the PET produced from the repurposed monomers.

Various chemical recycling technologies have been developed over the years. However, such processes typically make use of relatively hazardous chemicals and harsh reaction conditions. For example, common chemical recycling approaches are often performed under rather harsh and energy consuming conditions such as with high catalyst loadings and at high temperature and pressure.

Enzymatic depolymerisation of PET is a growing area of both academic and industrial interest. In a similar manner to chemical recycling, the enzymatic recycling pathway breaks down the PET back into building block monomers such as TPA and EG (or derivatives thereof).

While an enzymatic approach to recycling PET shows much promise, the enzymes used to date exhibit relatively poor activity against the highly crystalline form of most post-consumer PET waste. In an attempt to combat that problem, the PET must first be subjected to controlled thermal processing to render its crystalline structure amorphous, with the resulting amorphous PET then being subjected to the enzymatic hydrolysis. The need to amorphatise PET in advance of conventional enzymatic processing unfortunately makes the overall process less economically attractive on an industrial scale.

An opportunity therefore remains to develop new methodologies for recycling PET that address one or more of the problems associated with conventional recycling techniques, or at the least provides a useful alternative.

### SUMMARY OF THE INVENTION

The present invention provides a method for recycling polyethylene terephthalate, the method comprising:
(i) subjecting the PET to base-catalysed transesterification with a C₆-C₁₀ mono-alcohol to produce a composition comprising one or both of a mono-ester and diester terephthalate C₆-C₁₀ mono-alcohol derivative; and
(ii) converting the so formed one or both of mono-ester and diester terephthalate C₆-C₁₀ mono-alcohol derivative into terephthalic acid using an esterase.

Surprisingly, it has now been found that a specific transesterification reaction used in combination with an enzymatic process can be used to convert PET into terephthalic acid under mild conditions in a time efficient and economically viable manner. The so formed terephthalic acid can be reused in various applications, for example in the manufacture of PET, thereby enabling the feedstock PET of the method to be recycled.

The method in accordance with the invention can advantageously be scaled up into industrial production and can operate batch wise or continuously.

The method of the invention can also advantageously be used to separate and recycle PET from a mixture or blend of different materials.

In one embodiment, the PET that is to be recycled is provided in the form of a feedstock material comprising one or more co-materials that do not undergo base-catalysed transesterification with the C₆-C₁₀ mono-alcohol. According to that embodiment, after the PET has undergone the base-catalysed transesterification the PET becomes inherently separated from the one or more co-materials and thereby enables them to be readily recovered.

In one embodiment, the C₆-C₁₀ mono-alcohol is selected from hexanol, pentanol, octanol, nonanol, decanol and benzyl alcohol.

In another embodiment, the base catalysed transesterification reaction is performed at a temperature of less than 200° C., for example at a temperature ranging from about 100° C. to about 180° C.

In a one embodiment, the esterase is a polyesterase, diesterase or a monoesterase. In a further embodiment, the esterase is a PETase, a carboxylesterase or a MHETase.

In a further embodiment, the esterase is immobilised on a substrate and the method is performed continuously.

In another embodiment, the esterase is a) a PETase, wherein the PETase comprises an amino acid sequence of SEQ ID NO: 1 or an amino acid sequence having at least 70% sequence identity thereto; b) a carboxylesterase, wherein the carboxylesterase comprises an amino acid sequence of SEQ ID NO: 73, or an amino acid sequence that has at least 70% sequence identity to SEQ ID NO: 73; and/or c) a MHETase, wherein the MHETase comprises an amino acid that i) has at least 70% sequence identity to SEQ ID NO: 31, and ii) differs from SEQ ID NO:31 by an amino acid substitution at one or more positions selected from the group consisting of positions that correspond to amino acid positions 156 to 396, 398 to 410 and 425 to 603 of SEQ ID NO: 31. In a further embodiment, the esterase is: a PETase comprising the amino acid sequence of SEQ ID NO:29: a carboxylesterase comprising the amino acid sequence of SEQ ID NO: 73-77; and/or a MHETase comprising the amino acid sequence of SEQ ID NO:71.

The present invention further provides a polypeptide having esterase activity, wherein the polypeptide comprises an amino acid sequence of SEQ ID NO:29 or an amino acid sequence having at least 70% sequence identity thereto.

Further aspects and embodiments of the invention are discussed in more detail below.

### BRIEF DESCRIPTION OF DRAWINGS

FIG. 1. Illustrates results from Examples 1-2: A) Conversion (subtracting dry mass of remaining PET as % of original PET mass from 100%) following base-catalyzed transesterification in benzyl alcohol at various temperatures from 75-175° C. for 5 minutes. 100% conversion of PET is observed at 175° C. B) Conversion (subtracting dry mass of remaining PET as % of original PET mass from 100%) following base-catalyzed transesterification in various solvents for 5 minutes where the reaction temperature is just below the boiling temperature of the solvent. 100% conversion of PET is observed in 1-heptanol, 1-octanol and benzyl alcohol. C) Conversion of PET (%) following base-catalysed transesterification in benzyl alcohol for varying durations. 99.9% conversion of PET is observed at 15 minutes.

FIG. 2. Illustrates results from Example 4: Concentration (mg/mL) of TPA, monobenzyl terephthalate, and dibenzyl terephthalate over time for the base hydrolysis of product from the base-catalyzed transesterification of PET by benzyl alcohol.

FIG. 3. Illustrates results from Example 5: High performance liquid chromatography (HPLC) analysis of A) 0.2 mg/mL TPA standard (>99% purity, Sigma Aldrich) showing a retention time of 6.5 mins. B) Control incubation of 3.75 mM dibenzyl terephthalate in buffer (50 mM Bicine pH 9) at 60° C. for 1 hr shows no detectable TPA. C) Incubation of 3.75 mM dibenzyl terephthalate with 1 μM enzyme (SEQ ID NO:4; 1:3750 catalyst:substrate loading (molar ratio)) at 60° C. for 1 hr shows hydrolysis to TPA.

FIG. 4. Illustrates results from Example 6. Concentration of terephthalic acid (TPA) over time in the enzymatic hydrolysis of monobenzyl terephthalate, dibenzyl terephthalate, monooctyl terephthalate or dioctyl terephthalate (using engineered esterase corresponding to SEQ ID NO. 29). Concentrations of TPA were determined by analytical high-performance liquid chromatography (HPLC), using a calibration curve of TPA standards.

FIG. 5. Illustrates results from Example 8: High performance liquid chromatography (HPLC) analysis of A) 0.2 mg/mL TPA standard (>99% purity, Sigma Aldrich) showing a retention time of 6.5 min. B) 2% benzyl alcohol standard in 20 mM Bicine, 100 mM NaCl pH 9 buffer, showing a retention time of 7.6 min. C) 2% benzyl alcohol/monobenzyl terephthalate/TPA mixture in 20 mM Bicine, 100 mM NaCl pH 9 buffer, showing minimal absorbance at the retention time of TPA (6.5 min). D) 2% benzyl alcohol/monobenzyl terephthalate/TPA mixture in 20 mM Bicine, 100 mM NaCl pH 9 after hydrolysis by immobilized enzyme (SEQ ID NO: 29), showing increased concentration of TPA relative to C).

FIG. 6. Illustrates an alignment of homologous positions between an ancestrally reconstructed hydrolase having mono-, di- and poly-terephthalic esterase activity (SEQ ID NO: 4) and a variant thereof (SEQ ID NO:29) modified with amino acid substitutions F210V, N205C and S252C that improve activity for mono-di, and polyterephthalic esters, including improved mono-, di- and poly-terephthalic ester specificity when compared to the hydrolase of SEQ ID NO:4.

FIG. 7. Illustrates the results from Example 9. HPLC assay demonstrating the activity of MHETase of SEQ ID NO: 71 against the substrates monooctyl terephthalate (MOCT) and monobenzyl terephthalate (MBZT). A) The increase in TPA concentration over time compared to the control (no enzyme) when 200 nM of the polypeptide of SEQ ID NO: 71 was incubated with 1.5 mM MOCT at 40° C. is shown. B) The corresponding decrease in MOCT concentration is shown. The data demonstrate that all MOCT is converted to TPA within 8 minutes. C) The increase in TPA concentration over time compared to the control (no enzyme) when 200 nM of the polypeptide of SEQ ID NO:71 was incubated with 1.5 mM MBZT at 40° C. is shown. D) The corresponding decrease in MBZT concentration is shown. The data demonstrate that all MBZT is converted to TPA within 8 minutes.

FIG. 8. Illustrates the results from Example 10. Concentration of TPA (mg/mL) produced by carboxylesterases SEQ ID NOs: 73-77 after incubation with 1.5 mM MOCT at room temperature (23° C.) for 1 hour, using a 1:10 dilution of the eluate (containing purified protein) from Ni-NTA purification of the variants, or no enzyme (control). Concentrations of TPA were determined by analytical high-performance liquid chromatography (HPLC), using a calibration curve of TPA standards.

FIG. 9. Illustrates the results from Example 11. A) From left to right, the images depict white precipitated nylon in solution, nylon isolated by filtration, filtrate shows isolation of white dyes in top alcohol layer. B) 1H NMR in d₆-DMSO. Peak at 8.04 ppm is TPA, peak at 3.47 ppm is water, 2.5 is d₆-DMSO solvent. Peaks at 7.30-7.23 and 4.49 are benzyl alcohol.

### DETAILED DESCRIPTION OF THE INVENTION

The present invention provides a method for recycling PET. As used herein, the term “recycling” intended to mean using in the method of the invention PET that is no longer considered to be of practical use. While such PET will often be in the form of postconsumer waste, it may also be in the form of manufacturing waste or simply excess stock. According to the method the PET provides for a source of terephthalic acid (and other compounds such as ethylene glycol) that can be reused/recycled into various applications, for example to manufacture PET.

There is no particular limitation on the type of the PET that may be used in accordance with the invention. The PET may have any molecular weight and have a crystalline or amorphous morphology.

There is no particular limitation on the physical form of PET that may be used in accordance with the invention. To facilitate process handling and/or the rate of transesterification, it may be desirable for the PET to be in a comminuted form, for example in the form of PET flake or powder.

The PET may also be provided as a feedstock material in combination with one or more other co-materials. Those co-materials may or may not undergo reaction (e.g. transesterification) with the C₆-C₁₀ mono-alcohol.

Where such co-materials do not undergo reaction with the C₆-C₁₀ mono-alcohol the method can advantageously be used to separate the PET from those one or more other co-materials.

There is no particular limitation on the form and composition of the co-materials. For example, the one or more co-materials may be selected from metal, ceramic, glass, wood and polymer. In one embodiment, the one or more co-materials is a polymer selected from a polyamide (e.g. nylon), polyolefin (e.g. polyethylene and polypropylene) and polyvinylchloride (PVC).

The PET may be provided in mere admixture with the one or more co-materials, as a melt blend or even as a laminated structure.

The method in accordance with the invention comprises subjecting the PET to base-catalysed transesterification with a C₆-C₁₀ mono-alcohol. To perform that task, the PET and C₆-C₁₀ mono-alcohol will typically be contained within a reactor vessel.

Suitable reactor vessels include those used in conventional PET chemical recycling technologies such as reactor vessels made from glass or stainless steel. The volume of the reactor vessel will be tailored to meet the scale of the intended operation, such as lab scale, pilot scale through to industrial scale. If required, the reactor vessel may comprise one or more agitation or stirring devices to assist with movement and mixing of reagents within the reactor.

Those skilled in the art will appreciate transesterification of a polyester with an alcohol is often facilitated by use of a catalyst. Transesterification undertaken in accordance with the method of the invention makes use of a base catalyst. There is no particular limitation on the type of base catalyst that can be used.

In one embodiment, the transesterification is catalysed using an alkali metal base.

Examples of suitable alkali metal bases include, but are not limited to, alkali metal hydroxides.

Examples of suitable alkali metal hydroxides include, but are not limited to, lithium hydroxide, sodium hydroxide and potassium hydroxide.

In one embodiment, the transesterification is catalysed using sodium hydroxide or potassium hydroxide.

There is no particular limitation on the amount of base catalyst that can be used. Those skilled in the art can suitably select the amount of catalyst to use. For example, the amount of catalyst may be present in an amount ranging from 1:20 to 5:1 (mass of base catalyst:mass of PET).

The base catalyst may be combined with the other reaction components at any suitable time and in any suitable order, relative to the other reaction components.

The PET used in accordance with the invention undergoes transesterification with C₆-C₁₀ mono-alcohol. Those skilled in the art will appreciate such C₆-C₁₀ mono-alcohols are liquid at room temperature and therefore can conveniently be used as a reaction medium within which the transesterification reaction takes place. In other words, in addition to the C₆-C₁₀ mono-alcohol being used as a reactant it can also advantageously function in the method as a liquid reaction medium.

The C₆-C₁₀ mono-alcohol used in accordance with the invention will typically be present in a stoichiometric excess of that required to transesterify the entire mass of PET being used.

For example, the C₆-C₁₀ mono-alcohol may be used in at least a 2:1, or 3:1 or 4:1 excess to PET.

There is no particular limitation on the C₆-C₁₀ mono-alcohol that is used in accordance with the invention.

In one embodiment, the C₆-C₁₀ mono-alcohol is selected from hexanol, pentanol, octanol, nonanol, decanol and benzylalcohol.

In a further embodiment, the C₆-C₁₀ mono-alcohol is selected from octanol and benzylalcohol.

The method may be performed using a combination of two or more C₆-C₁₀ mono-alcohols.

While not required, if desired the transesterification may be performed in the presence of one or more other liquid reaction media, for example an inert solvent. By “inert solvent” is meant a liquid that does not adversely affect or take part in the transesterification reaction. Examples of such other liquid reaction media include, but are not limited to, dimethyl sulfoxide (DMSO), dimethyl formamide (DMF), Cyrene™ and ethylene glycol.

Provided the PET, base catalyst and C₆-C₁₀ mono-alcohol make suitable contact to undergo transesterification, there is no particular limitation on how each component is introduced into a suitable reactor vessel. For example, the PET, base catalyst and C₆-C₁₀ mono-alcohol may be introduced to the vessel in combination or individually.

As described in more detail below, the method of the invention may be performed continuously and thereby require continuous addition of one or more feedstock components.

The transesterification reaction that takes place in accordance with the invention produces a composition comprising one or both of a mono-ester and diester terephthalate C₆-C₁₀ mono-alcohol derivative. The transesterification reaction may therefore be schematically represented below in reaction scheme 1.

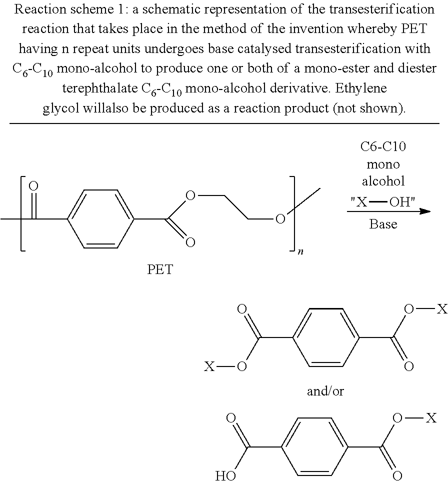

Accordingly, it will be appreciated reference herein to the so formed “mono-ester and diester terephthalate C₆-C₁₀ mono-alcohol derivative” is intended to mean the mono-ester and diester terephthalate compounds formed through transesterification of the PET with C₆-C₁₀ mono-alcohol.

One or more other reaction products (e.g. ethylene glycol, disodium terephthalic acid salt) form during the transesterification reaction, but the mono-ester and diester terephthalate C₆-C₁₀ mono-alcohol derivatives are of particular relevance to the present invention. The mono-ester and diester terephthalate C₆-C₁₀ mono-alcohol derivatives will typically be major target products of the transesterification reaction.

The transesterification reaction depicted in scheme I can advantageously be performed under relatively mild reaction conditions and proceeds with high conversion.

In one embodiment, the base catalysed transesterification is performed at a temperature of less than 200° C., for example of less than about 190° C., or less than about 180° C., or less than about 170° C., or less than about 160° C., or less than about 150° C.

In another embodiment, the base catalysed transesterification is performed at a temperature of greater than about 100° C., for example greater than about 110° C., or greater than about 120° C., or greater than about 130° C., or greater than about 140° C., or greater than about 150° C., or greater than about 160° C.

In a further embodiment, the base catalysed transesterification is performed at a temperature ranging from about 75° C. to about 200° C., or from about 75° C. to about 180° C., or from about 100° C. to about 180° C., or from about 130° C. to about 175° C.

Heat may be applied to the transesterification reaction using conventional means.

For example, the reaction components may be combined in a reactor vessel and heat applied to the reactor vessel to increase the temperature of the reaction components to a desired temperature. Alternatively, the C₆-C₁₀ mono-alcohol may be heated to a desired temperature and combined with the other reaction components.

In one embodiment, the PET, base catalyst and C₆-C₁₀ mono-alcohol are combined and heated to a transesterification reaction temperature as described herein (for example to at least 100° C.).

In a further embodiment, the C₆-C₁₀ mono-alcohol is heated to a transesterification reaction temperature as described herein (for example to at least 100° C.) and then combined with the PET and base catalyst.

The transesterification can be performed at atmospheric pressure or pressures up to 5 atm.

In one embodiment, the transesterification is performed at atmospheric pressure.

The transesterification reaction is conducted for a period of time suitable to produce the required one or both mono-ester and diester terephthalate C₆-C₁₀ mono-alcohol derivatives. The required time will of course depend on variables such as temperature, pressure, surface area of the PET and the mass ratio of the base catalyst:PET:C₆-C₁₀ mono-alcohol.

The transesterification reaction can be performed in a time efficient manner.

Advantageously, the transesterification can proceed quite rapidly. For example, the transesterification may take place with high conversion over a period of time ranging from only about 5 mins to about 1 hour.

Transesterification of the PET in accordance with the invention produces a composition comprising one or both of a mono-ester and diester terephthalate C₆-C₁₀ mono-alcohol derivative.

In one embodiment, the transesterification produces a composition comprising a diester terephthalate C₆-C₁₀ mono-alcohol derivative.

In a further embodiment, the transesterification produces a composition comprising a mono-ester terephthalate C₆-C₁₀ mono-alcohol derivative.

In another embodiment, the transesterification produces a composition comprising both mono-ester and diester terephthalate C₆-C₁₀ mono-alcohol derivatives.

The formation of diester terephthalate C₆-C₁₀ mono-alcohol derivative will generally be favoured when the C₆-C₁₀ mono-alcohol is used in stoichiometric excess relative to the amount of PET present.

As will be discussed in more detail below, the amount of mono-ester terephthalate C₆-C₁₀ mono-alcohol derivative produced can be enhanced by subjecting the transesterification reaction products to an alkaline aqueous treatment.

The composition comprising one or both of a mono-ester and diester terephthalate C₆-C₁₀ mono-alcohol derivative will generally also comprise the relevant C₆-C₁₀ mono-alcohol and one or more other reaction by products.

Before proceeding with remaining step (ii) of the method, it may be desirable to subject the composition produced from the transesterification reaction to one or more intermediate process steps. For example, such intermediate process steps can serve to (i) increase purity of the so formed one or both of a mono-ester and diester terephthalate C₆-C₁₀ mono-alcohol derivatives, and/or (ii) promote conversion of any diester terephthalate C₆-C₁₀ mono-alcohol derivative present into mono-ester terephthalate C₆-C₁₀ mono-alcohol derivative, with the resulting composition comprising one or both of a mono-ester and diester terephthalate C₆-C₁₀ mono-alcohol derivatives to be used in step (ii).

In one embodiment, prior to undertaking step (ii) the composition produced in step (i) is processed to increase purity of the so formed one or both of a mono-ester and diester terephthalate C₆-C₁₀ mono-alcohol derivatives, with the resulting processed composition being used in step (ii).

In another embodiment, prior to undertaking step (ii) the composition produced in step (i) is processed to convert some or all of any diester terephthalate C₆-C₁₀ mono-alcohol derivative present into mono-ester terephthalate C₆-C₁₀ mono-alcohol derivative, with the resulting processed composition being used in step (ii).

The composition produced in step (i) may be processed to increase the purity of the mono-ester/diester terephthalate C₆-C₁₀ mono-alcohol derivatives using purification techniques well-known to those skilled in the art. For example, the composition may be subjected to one or more techniques selected from solvent extraction, solvent washing, filtration, distillation, solvent evaporation, column chromatography and crystallisation.

In one embodiment, prior to undertaking step (ii) the composition produced in step (i) is processed to increase the purity of the so formed one or both of a mono-ester and diester terephthalate C₆-C₁₀ mono-alcohol derivatives using one or more techniques selected from solvent extraction, solvent washing, filtration, distillation, solvent evaporation, column chromatography and crystallisation.

For example, prior to undertaking step (ii) the composition produced in step (i) may be washed with water to increase the purity of the so formed one or both of mono-ester and diester terephthalate C₆-C₁₀ mono-alcohol derivatives. Such a washing step may assist with removing compounds such as ethylene glycol and/or disodium terephthalic acid produced in step (i).

In a further embodiment, mono-ester and/or diester terephthalate C₆-C₁₀ mono-alcohol derivative present in the composition produced in step (i) is purified and isolated prior to it being used in step (ii).

Where the composition produced in step (i) is processed by subjecting it to solvent washing using an alkaline aqueous composition it has been found the alkaline aqueous composition not only extracts reaction byproducts such as ethylene glycol from the esterification composition (to thereby purify the mono-ester and/or diester terephthalate C₆-C₁₀ mono-alcohol derivative), but also promotes hydrolysis of any diester terephthalate C₆-C₁₀ mono-alcohol derivative present into mono-ester terephthalate C₆-C₁₀ mono-alcohol derivative. Accordingly, such an alkaline aqueous solvent wash process step advantageously not only purifies the esterification composition produced in step (i) but also facilitates conversion of diester terephthalate C₆-C₁₀ mono-alcohol derivative into mono-ester terephthalate C₆-C₁₀ mono-alcohol derivative for use in step (ii).

In one embodiment, prior to undertaking step (ii) the composition produced in step (i) is washed with an aqueous alkaline composition to produce an aqueous phase and a non-aqueous phase comprising one or both of a mono-ester and diester terephthalate C₆-C₁₀ mono-alcohol derivative, with the resulting non-aqueous phase being used in step (ii)

Washing of the composition produced in step (i) with an alkaline aqueous composition may be performed at temperatures ranging from room temperature to about 90° C., for example from about 25° C. to about 90° C., or from about 40° C. to about 90° C., or from about 60° C. to about 90° C.

There is no particular limitation on the volume of alkaline aqueous composition used relative to the volume of the composition produced in step (i). However, the volume of alkaline aqueous composition to the composition produced in step (i) will generally range from about 1:1 to about 1:5, for example from about 1:2 to about 1:4, or about 1:3.

The alkaline aqueous composition will generally be provided by combining an alkaline metal hydroxide with water. Suitable alkaline metal hydroxides include, but are not limited to, sodium hydroxide and potassium hydroxide.

Production of the one or both of a mono-ester and diester terephthalate C₆-C₁₀ mono-alcohol derivative can be confirmed using techniques well-known to those skilled in the art. For example, the presence of a mono-ester and diester terephthalate C₆-C₁₀ mono-alcohol derivative can be readily determined using NMR spectroscopy.

Where the PET used in accordance with the invention is provided in the form of feedstock material also comprising one or more other co-materials that do not undergo reaction with the C₆-C₁₀ mono-alcohol, the method can also advantageously be used to separate the PET from the one or more other co-materials. For example, a number of commercial products are provided in a form made up of multiple different polymers, including PET. Many polymer recycling operations are not suitable for processing mixed polymer feedstock as they are typically tailored for processing only a single class of polymer. There are many polymers that are likely to be present with the PET in such products that will not undergo reaction (e.g. base-catalysed transesterification) with the C₆-C₁₀ mono-alcohol and for the most part will remain intact upon being processed in the method of the invention. Accordingly, the method of the invention can advantageously be used as a means for separating PET from other materials, including other polymers.

In one embodiment, the PET that is to be recycled is provided in the form of a feedstock material comprising one or more co-materials that do not undergo reaction with the C₆-C₁₀ mono-alcohol.

According to that embodiment, after the PET has undergone the base-catalysed transesterification the PET becomes inherently separated from the one or more co-materials and thereby enables them to be readily recovered. For example, the feedstock material for use in accordance with the invention may be in the form of a PET/PVC laminate. Such laminates are often difficult to separate and conventional recycling techniques are unsuitable due to the presence of the two different polymers. The method according to the present invention can not only take such a PET/PVC laminate and seek to recycle the PET component, but the process inherently separates the PET from the PVC. In that regard, according to the method the PET undergoes transesterification with the C₆-C₁₀ mono-alcohol, the process of which effectively separates the PET from the PVC (which does not undergo reaction with the C₆-C₁₀ mono-alcohol). After that transesterification step, the PVC can be isolated and itself recycled.

Where the PET is provided in the form of a feedstock material comprising one or more co-materials that will not undergo reaction with the C₆-C₁₀ mono-alcohol, the PET will inherently become separated from the one or more co-materials after it has undergone base catalysed transesterification with the C₆-C₁₀ mono-alcohol. The one or more co-materials can then be isolated for subsequent use.

In one embodiment, the PET is provided in the form of a laminate with one or more other polymers that do not undergo reaction with the C₆-C₁₀ mono-alcohol.

Step (ii) in accordance with the method of the invention comprises converting the one or both of a mono-ester and diester terephthalate C₆-C₁₀ mono-alcohol derivative produced in step (i) into terephthalic acid using an esterase. The enzymatic process of step (ii) may be schematically represented below in reaction scheme 2.

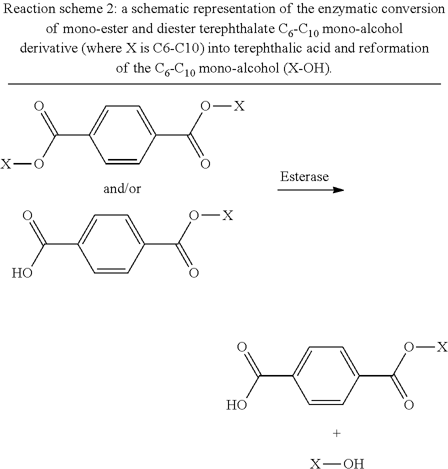

It has surprisingly been found the enzymatic conversion of such mono-ester and diester terephthalate C₆-C₁₀ mono-alcohol derivatives occurs rapidly and efficiently relative to conventional PET enzymatic degradation processes.

The enzymatic conversion step may be performed using techniques and equipment known to those skilled in the art.

Generally, the composition comprising the one or both of a mono-ester and diester terephthalate C₆-C₁₀ mono-alcohol derivative will be provided in combination with a solvent and the esterase in a reactor vessel and incubated for a suitable period of time for undertaking conversion into the terephthalic acid.

In one embodiment, step (ii) comprises providing the one or both of a mono-ester and diester terephthalate C₆-C₁₀ mono-alcohol derivative with a solvent and the esterase in a reactor vessel and incubating the so formed composition for a suitable period of time for undertaking conversion into the terephthalic acid.

Examples of suitable solvents that may be used in the enzymatic conversion include, but are not limited to, C₆-C₁₀ mono-alcohol, dimethyl sulfoxide (DMSO), methanol and dimethyl formamide (DMF).

In one embodiment, the solvent used in step (ii) comprises C₆-C₁₀ mono-alcohol from step (i).

The time taken for conversion of the one or both of a mono-ester and diester terephthalate C₆-C₁₀ mono-alcohol derivatives into terephthalic acid using the esterase will vary depending upon at least temperature and the ratio of enzyme to transesterification product. Generally, conversion of the one or both of a mono-ester and diester terephthalate C₆-C₁₀ mono-alcohol derivatives into terephthalic acid using the esterase will require an incubation period ranging from about 5 mins to about 24 hours. Generally, the incubation period may range from about 5 minutes to about 20 hours, from about 5 minutes to about 18 hours, from about 5 minutes to about 16 hours, from about 5 minutes to about 14 hours, from about 5 minutes to about 12 hours, from about 5 minutes to about 10 hours, from about 5 minutes to about 8 hours, from about 5 minutes to about 6 hours. In further examples, the incubation period may be from about 5 minutes to about 5 hours, 5 minutes to about 4 hours, from about 5 minutes to about 3 hours, from about 5 minutes to about 2 hours, and from about 5 minutes to about 1 hour.

Those skilled in the art will be able to select a suitable incubation temperature for use with the particular esterase. Generally, incubation may be performed at a temperature ranging from about 0° C. to about 80° C., for example from about 2° C. to 75° C., from about 4° C. to 70° C., from about 6° C. to 70° C.: from about 8° C. to 65° C., from about 10° C. to 65° C., from about 20° C. to 60° C., from about 25° C. to 55° C. from about 30° C. to 55° C., from about 30° C. to 50° C.

The esterase used in accordance with invention may be a polyesterase, diesterase or monesterase.

The term “esterase” typically refers to a hydrolase enzyme classified as EC 3.1 according to Enzyme Nomenclature and catalyses the hydrolysis of ester bonds to produce an acid and an alcohol. Herein, the esterase will suitably catalyse the hydrolysis of mono- and di-, and/or poly-terephthalic acid esters.

In one embodiment, the esterase includes PETase, carboxylesterases, MHETases and cutinases. In an embodiment, the esterase is a poly(ethylene terephthalate) hydrolase (PETase). PETases are typically classified as EC 3.1.1.101 according to Enzyme Nomenclature and catalyze the hydrolysis of polyethylene terephthalate (PET) plastic to monomeric mono-2-hydroxyethyl terephthalate (MHET).

Suitable PETases will be familiar to persons skilled in the art, an illustrative example of which includes the wild-type Ideonella sakaiensis PETase (strain NBRC 110686/TISTR 2288/201-F6), as described by Yoshida et al. (2016, Science, 352 (6278): 1196), the contents of which are incorporated herein by reference in their entirety.

In an embodiment, the esterase comprises an amino acid sequence of SEQ ID NO: 1 or an amino acid sequence having at least 70% sequence identity thereto.

(UniProt Accession No. A0A0K8P6T7-1)
SEQ ID NO: 1
MNFPRASRLMQAAVLGGLMAVSAAATAQTNPYARGPNPTAASLEASAGP
FTVRSFTVSRPSGYGAGTVYYPTNAGGTVGAIAIVPGYTARQSSIKWWG
PRLASHGFVVITIDTNSTLDQPSSRSSQQMAALRQVASLNGTSSSPIYG
KVDTARMGVMGWSMGGGGSLISAANNPSLKAAAPQAPWDSSTNFSSVTV
PTLIFACENDSIAPVNSSALPIYDSMSRNAKQFLEINGGSHSCANSGNS
NQALIGKKGVAWMKRFMDNDTRYSTFACENPNSTRVSDFRTANCS

By “at least 70%” is meant that the esterase shares at least 70%, preferably at least 75%, preferably at least 80%, preferably at least 85%, preferably at least 90%, preferably at least 92%, preferably at least 94%, preferably at least 95%, preferably at least 96%, preferably at least 97%, preferably at least 98%, or more preferably at least 99% sequence identity to SEQ ID NO: 1. In an embodiment, the esterase comprises an amino acid sequence that differs from SEQ ID NO: 1 by one or more amino acid insertions, substitutions and/or deletions, such as at the N- and/or C-termini, as long as the esterase retains at least some hydrolase activity, such as when compared to an extant esterase, including the PETase of SEQ ID NO: 1. In an embodiment, the esterase may comprise a modification or alteration at one or more (e.g., several) amino acid positions that enhances its activity in catalysing the hydrolysis of mono- and di-, and poly-terephthalic acid esters, such as when compared to an extant esterase, including the PETase of SEQ ID NO:1. Esterases that comprise an amino acid sequence that differs from the amino acid sequence of extant or wild-type esterase (e.g., the wild-type PETase of SEQ ID NO: 1) may be referred to as variants or functional variants. Such variants can be obtained by techniques known to persons skilled in the art, illustrative examples of which include site-directed mutagenesis, random mutagenesis and synthetic oligonucleotide construction.

The terms “modification”, “alteration”, “substitution” and the like, as used herein in relation to an amino acid residue or position, typically mean that the amino acid in the particular position has been modified compared to the amino acid of the wild-type or reference esterase. Suitable substitutions may include the replacement of an amino acid residue by another selected from the naturally-occurring standard 20 amino acid residues, rare naturally occurring amino acid residues (e.g., hydroxyproline, hydroxylysine, allohydroxylysine, 6-N-methylysine, N-ethylglycine, N-methylglycine, N-ethylasparagine, allo-isoleucine, N-methylisoleucine, N-methylvaline, pyroglutamine, aminobutyric acid, ornithine, norleucine, norvaline), and non-naturally occurring amino acid residue, often made synthetically, (e.g., cyclohexyl-alanine). Preferably, the substitution comprises the replacement of an amino acid residue by another selected from the naturally-occurring standard 20 amino acid residues (G, P, A, V, L, I, M, C, F, Y, W, H, K, R, Q, N, E, D, S and T). The substitution can be a conservative or non-conservative substitution. Examples of conservative substitutions will be familiar to persons skilled in the art, illustrative examples of which include substitutions within the groups of basic amino acids (arginine, lysine and histidine), acidic amino acids (glutamic acid and aspartic acid), polar amino acids (glutamine, asparagine and threonine), hydrophobic amino acids (methionine, leucine, isoleucine, cysteine and valine), aromatic amino acids (phenylalanine, tryptophan and tyrosine), and small amino acids (glycine, alanine and serine).

In an embodiment, the esterase comprises an amino acid sequence selected from the group consisting of SEQ ID NOs: 2-29 and an amino acid sequence having at least 70% sequence identity to any of the foregoing.

In an embodiment, the esterase comprises an amino acid sequence of SEQ ID NO:4 or SEQ ID NO: 29, or an amino acid sequence having at least 70% sequence identity to any of the foregoing.

In another aspect disclosed herein, there is provided a polypeptide having esterase activity, wherein the polypeptide comprises an amino acid sequence of SEQ ID NO:29 or an amino acid sequence having at least 70% sequence identity thereto.

In an embodiment, the polypeptide comprises an amino acid sequence of SEQ ID NO:29 or an amino acid sequence having at least 77% sequence identity thereto.

In an embodiment, the polypeptide comprises an amino acid sequence of SEQ ID NO:29.

In an embodiment, the esterase is a MHETase. MHETase enzymes are typically classified as EC 3.1.1.102 according to NC-IUBMB Enzyme Nomenclature. MHETase enzymes catalyse the hydrolysis of the monoester terephthalate mono-(2-hydroxyethyl) terephthalate (MHET, which is produced by PETase hydrolysis of PET) into terephthalate and ethylene glycol. In some embodiments, the MHETase enzyme is capable of hydrolysis of other monoester terephthalates.

In some embodiments, the MHETase is capable of hydrolysis of mono-ester terephthalate C₆-C₁₀ mono-alcohol derivatives into terephthalic acid. In an embodiment, the MHETase is capable of hydrolysis of monobenzyl terephthalate (MBZT), monohexyl terephthalate, monoheptyl terephthalate (MHPT) and/or monooctyl terephthalate (MOCT) into terephthalic acid. In a preferred embodiment, the MHETase is capable of hydrolysis of monobenzyl terephthalate (MBZT) into terephthalic acid. In a preferred embodiment, the MHETase is capable of hydrolysis of monooctyl terephthalate (MOCT) into terephthalic acid.

In an embodiment, the esterase comprises an amino acid sequence that (i) has at least 70% sequence identity to SEQ ID NO: 31 and (ii) differs from SEQ ID NO:31 by an amino acid substitution at one or more positions selected from the group consisting of positions that correspond to amino acid positions 156 to 396, 398 to 410 and 425 to 603 of SEQ ID NO:31.

(UniProt Accession No. A0A0K8P8E7)
SEQ ID NO: 31
GGGSTPLPLPQQQPPQQEPPPPPVPLASRAACEALKDGNGDMVWPNAAT
VVEVAAWRDAAPATASAAALPEHCEVSGAIAKRTGIDGYPYEIKFRLRM
PAEWNGRFFMEGGSGTNGSLSAATGSIGGGQIASALSRNFATIATDGGH
DNAVNDNPDALGTVAFGLDPQARLDMGYNSYDQVTQAGKAAVARFYGRA
ADKSYFIGCSEGGREGMMLSQRFPSHYDGIVAGAPGYQLPKAGISGAWT
TQSLAPAAVGLDAQGVPLINKSFSDADLHLLSQAILGTCDALDGLADGI
VDNYRACQAAFDPATAANPANGQALQCVGAKTADCLSPVQVTAIKRAMA
GPVNSAGTPLYNRWAWDAGMSGLSGTTYNQGWRSWWLGSFNSSANNAQR
VSGFSARSWLVDFATPPEPMPMTQVAARMMKFDFDIDPLKIWATSGQFT
QSSMDWHGATSTDLAAFRDRGGKMILYHGMSDAAFSALDTADYYERLGA
AMPGAAGFARLFLVPGMNHCSGGPGTDRFDMLTPLVAWVERGEAPDQIS
AWSGTPGYFGVAARTRPLCPYPQIARYKGSGDINTEANFACAAPP

In an embodiment, the esterase comprises an amino acid substitution at one or more positions selected from the group consisting of
(i) a position that corresponds to amino acid positions 156 of SEQ ID NO:31;
(ii) a position that corresponds to amino acid position 159 of SEQ ID NO:31;
(iii) a position that corresponds to amino acid position 192 of SEQ ID NO:31;
(iv) a position that corresponds to amino acid position 196 of SEQ ID NO:31;
(v) a position that corresponds to amino acid position 197 of SEQ ID NO:31;
(vi) a position that corresponds to amino acid position 252 of SEQ ID NO:31;
(vii) a position that corresponds to amino acid position 260 of SEQ ID NO:31;
(viii) a position that corresponds to amino acid position 264 of SEQ ID NO:31;
(ix) a position that corresponds to amino acid position 267 of SEQ ID NO:31;
(x) a position that corresponds to amino acid position 286 of SEQ ID NO:31; and
(xi) a position that corresponds to amino acid position 503 of SEQ ID NO: 31.

In an embodiment, the esterase comprises an amino acid sequence that differs from SEQ ID NO: 31 by amino acid substitutions at positions that correspond to amino acid positions 156 and 159 of SEQ ID NO:31. In another embodiment, the esterase comprises an amino acid sequence that differs from SEQ ID NO: 31 by the amino acid substitutions N156G, T159V or a conservative amino acid substitution thereof.

In an embodiment, the esterase comprises an amino acid sequence that differs from SEQ ID NO: 31 by amino acid substitutions at positions that correspond to amino acid positions 156, 159, and 503 of SEQ ID NO:31. In another embodiment, the esterase comprises an amino acid sequence that differs from SEQ ID NO: 31 by the amino acid substitutions N156G, T159V and Y503W, or a conservative amino acid substitution thereof.

In an embodiment, the esterase comprises an amino acid sequence that differs from SEQ ID NO: 31 by amino acid substitutions at positions that correspond to amino acid positions 156, 159, 192 and 503 of SEQ ID NO:31. In another embodiment, the esterase comprises an amino acid sequence that differs from SEQ ID NO: 31 by the amino acid substitutions N156G, T159V, M192Y and Y503W, or a conservative amino acid substitution thereof.

In an embodiment, the esterase comprises an amino acid sequence that differs from SEQ ID NO: 31 by amino acid substitutions at positions that correspond to amino acid positions 159, 252 and 503 of SEQ ID NO:31. In another embodiment, the esterase comprises an amino acid sequence that differs from SEQ ID NO: 31 by the amino acid substitutions T159V, Y252F and Y503W, or a conservative amino acid substitution thereof.

In an embodiment, the esterase comprises an amino acid sequence that differs from SEQ ID NO: 31 by amino acid substitutions at positions that correspond to amino acid positions 159, 192, 252 and 503 of SEQ ID NO:31. In another embodiment, the esterase comprises an amino acid sequence that differs from SEQ ID NO: 31 by the amino acid substitutions T159V, M192Y, Y252F and Y503W, or a conservative amino acid substitution thereof.

In an embodiment, the esterase comprises an amino acid sequence that differs from SEQ ID NO: 31 by amino acid substitutions at positions that correspond to amino acid positions 159, 192 and 503 of SEQ ID NO:31. In another embodiment, the esterase comprises an amino acid sequence that differs from SEQ ID NO: 31 by the amino acid substitutions T159V, M192Y and Y503W, or a conservative amino acid substitution thereof.

In one embodiment, the esterase comprises an amino acid sequence selected from the group consisting of SEQ ID NOs: 31-72, or an amino acid sequence having at least 70% sequence identity to any of the foregoing.

In another embodiment, the esterase comprises an amino acid sequence of SEQ ID NO:71.

In an embodiment, the esterase is a carboxylesterase. Carboxylesterases are typically classified as EC 3.1.1.1 according to NC-IUBMB Enzyme Nomenclature.

In some embodiments, the carboxylesterase is capable of hydrolysis of mono- and di-, and/or poly-terephthalic acid esters. In some embodiments, the carboxylesterase is capable of hydrolysis of mono- and di-terephthalate C₆-C₁₀ mono-alcohol derivatives into terephthalic acid. In another embodiment the carboxylesterase is capable of hydrolysis of monobenzyl terephthalate (MBZT), monohexyl terephthalate, monoheptyl terephthalate (MHPT) and/or monooctyl terephthalate (MOCT), dibenzyl terephthalate (DBZT), dihexyl terephthalate, diheptyl terephthalate (DHPT) and/or dioctyl terephthalate (DOCT) into terephthalic acid. In a preferred embodiment, the carboxylesterase is capable of hydrolysis of MBZT and/or DBZT into terephthalic acid. In a preferred embodiment, the carboxylesterase is capable of hydrolysis of MOCT and/or DOCT into terephthalic acid.

In an embodiment, the esterase comprises an amino acid sequence of SEQ ID NO: 73, or an amino acid sequence that has at least 70% sequence identity to SEQ ID NO: 73.

SEQ ID NO: 73
MLLPETRNLLDLMDAATRGGRPRLETLPHAVGRKAVDKMSEDGEADPPE
VAEVANGGFAGPASEIRFRRYRPLGEAAGLLPTLIYYHGGGFVIGNIET
HDSTCRRLANKSRCQVISIDYRLAPEHPFPAPIDDGIAAFRHIRDNAES
FGADAARLAVGGDSAGGAMAAVVCQACRDAGETGPAFQMLIYPATDSSR
ESASRVAFAEGYFLSKALMDWFWEAYVPEDTDLTDLRLSPLLATDFTGL
PPAFVLTAGYDPLRDEGRAYADRLIEAGIKTTYVNYPGTIHGFFSLTRF
LSQGLKANDEAAAVMGAHFGT*

By “at least 70%” is meant that the esterase shares at least 70%, preferably at least 75%, preferably at least 80%, preferably at least 85%, preferably at least 90%, preferably at least 92%, preferably at least 94%, preferably at least 95%, preferably at least 96%, preferably at least 97%, preferably at least 98%, or more preferably at least 99% sequence identity to SEQ ID NO: 73. In an embodiment, the esterase comprises an amino acid sequence that differs from SEQ ID NO: 73 by one or more amino acid insertions, substitutions and/or deletions, such as at the N- and/or C-termini, as long as the esterase retains at least some esterase activity, such as when compared to an extant esterase, including the carboxylesterase of SEQ ID NO:73.

In an embodiment, the esterase may comprise a modification or alteration at one or more (e.g., several) amino acid positions that enhances its activity in catalysing the hydrolysis of mono- and di-, and poly-terephthalic acid esters, such as when compared to an extant esterase, including the carboxylesterase of SEQ ID NO:73. Esterases that comprise an amino acid sequence that differs from the amino acid sequence of extant or wild-type esterase (e.g., the wild-type carboxylesterase of SEQ ID NO:73) may be referred to as variants or functional variants. Such variants can be obtained by techniques known to persons skilled in the art, illustrative examples of which include site-directed mutagenesis, random mutagenesis and synthetic oligonucleotide construction.

In one embodiment, the esterase comprises an amino acid sequence selected from the group consisting of SEQ ID NOs: 73-77, or an amino acid sequence having at least 70% sequence identity to any of the foregoing.

TABLE 1
Amino acid sequences of esterase enzymes

| SEQ | |
|---|---|
| ID | |
| NO: | Amino acid sequence |
| 1 | MNFPRASRLMQAAVLGGLMAVSAAATAQTNPYARGPNPTA |
| | ASLEASAGPFTVRSFTVSRPSGYGAGTVYYPTNAGGTVGAIAI |
| | VPGYTARQSSIKWWGPRLASHGFVVITIDTNSTLDQPSSRSSQ |
| | QMAALRQVASLNGTSSSPIYGKVDTARMGVMGWSMGGGGS |
| | LISAANNPSLKAAAPQAPWDSSTNFSSVTVPTLIFACENDSIAP |
| | VNSSALPIYDSMSRNAKQFLEINGGSHSCANSGNSNQALIGKK |
| | GVAWMKRFMDNDTRYSTFACENPNSTRVSDFRTANCS |
| 2 | MYERGPDPTEASLEASSGPFSVSETSVSRLSASGFGGGTIYYPT |
| | TTSSGTYGAVAISPGYTATQSSIAWLGPRLASHGFVVITIDTNT |
| | TFDQPDSRARQLMAALNYLVNRSSVRSRIDSSRLAVMGHSM |
| | GGGGTLRAAEDNPSLKAAIPLTPWHTNKNWSSVRVPTLIIGAE |
| | NDTIAPVSSHAKPFYNSLPSSTPKAYLELNGASHFAPNSSNTTI |
| | GKYSVSWLKRFVDNDTRYSQFLCPAPHDDSAISEYRSTCPY |
| 3 | MYERGPDPTESSLEASSGPFSVSETSVSRLSASGFGGGTIYYPT |
| | TTSEGTYGAVAISPGYTATQSSIAWLGPRLASHGFVVITIDTNT |
| | TFDQPDSRARQLMAALNYLVNRSSVRSRIDSSRLAVMGHSM |
| | GGGGTLRAAEDNPSLKAAIPLTPWHTNKNWSSVRVPTLIIGAE |
| | NDTIAPVSSHAKPFYNSLPSSTPKAYLELNGASHFAPNSSNTTI |
| | GKYSIAWLKRFVDNDTRYSQFLCPAPHDDSAISEYRSTCPY |
| 4 | AANPYERGPDPTEASLEASSGPFSVSETSVSRLSASGFGGGTIY |
| | YPTTTSSGTYGAVAISPGYTATQSSIAWLGPRLASHGFVVITID |
| | TNTTFDQPDSRARQLMAALNYLVNRSSVRSRIDSSRLAVMGH |
| | SMGGGGTLRAAEDNPSLKAAIPLTPWHTNKNWSSVRVPTLII |
| | GAENDTIAPVSSHAKPFYNSLPSSTPKAYLELNGASHFAPNSS |
| | NTTIGKYSVSWLKRFVDNDTRYSQFLCPAPHDDSAISEYRSTC |
| | PY |
| 5 | AANPYERGPDPTESSLEASSGPFSVSETSVSRLSASGFGGGTIY |
| | YPTTTSEGTYGAVAISPGYTATQSSIAWLGPRLASHGFVVITID |
| | TNTTFDQPDSRARQLMAALNYLVNRSSVRSRIDSSRLAVMGH |
| | SMGGGGTLRAAEDNPSLKAAIPLTPWHTNKNWSSVRVPTLII |
| | GAENDTIAPVSSHAKPFYNSLPSSTPKAYLELNGASHFAPNSS |
| | NTTIGKYSIAWLKRFVDNDTRYSQFLCPAPHDDSAISEYRSTC |
| | PY |
| 6 | ADNPYERGPDPTEASLEASSGPFSVSETSVSRLSASGFGGGTIY |
| | YPTTTSSGTYGAVAISPGYTATQSSIAWLGPRLASHGFVVITID |
| | TNTTFDQPDSRARQLMAALNYLVNRSSVRSRIDSSRLAVMGH |
| | SMGGGGTLRAAEDNPSLKAAIPLTPWHTNKNWSSVRVPTLII |
| | GAENDTIAPVSSHAKPFYNSLPSSTPKAYLELNGASHFAPNSS |
| | NTTIGKYSVSWLKRFVDNDTRYSQFLCPAPHDDSAISEYRSTC |
| | PY |
| 7 | AANPYERGPEPTEASLEASSGPFSVSETSVSRLSASGFGGGTIY |
| | YPTTTSSGTYGAVAISPGYTATQSSIAWLGPRLASHGFVVITID |
| | TNTTFDQPDSRARQLMAALNYLVNRSSVRSRIDSSRLAVMGH |
| | SMGGGGTLRAAEDNPSLKAAIPLTPWHTNKNWSSVRVPTLII |
| | GAENDTIAPVSSHAKPFYNSLPSSTPKAYLELNGASHFAPNSS |
| | NTTIGKYSVSWLKRFVDNDTRYSQFLCPAPHDDSAISEYRSTC |
| | PY |
| 8 | AANPYERGPDPTEASVEASSGPFSVSETSVSRLSASGFGGGTIY |
| | YPTTTSSGTYGAVAISPGYTATQSSIAWLGPRLASHGFVVITID |
| | TNTTFDQPDSRARQLMAALNYLVNRSSVRSRIDSSRLAVMGH |
| | SMGGGGTLRAAEDNPSLKAAIPLTPWHTNKNWSSVRVPTLII |
| | GAENDTIAPVSSHAKPFYNSLPSSTPKAYLELNGASHFAPNSS |
| | NTTIGKYSVSWLKRFVDNDTRYSQFLCPAPHDDSAISEYRSTC |
| | PY |
| 9 | AANPYERGPDPTEASLEASSGPFSVSETSVSRLSASGFGGGTIY |
| | YPTTTSAGTYGAVAISPGYTATQSSIAWLGPRLASHGFVVITID |
| | TNTTFDQPDSRARQLMAALNYLVNRSSVRSRIDSSRLAVMGH |
| | SMGGGGTLRAAEDNPSLKAAIPLTPWHTNKNWSSVRVPTLII |
| | GAENDTIAPVSSHAKPFYNSLPSSTPKAYLELNGASHFAPNSS |
| | NTTIGKYSVSWLKRFVDNDTRYSQFLCPAPHDDSAISEYRSTC |
| | PY |
| 10 | AANPYERGPDPTEASLEASSGPFSVSETSVSRLSASGFGGGTIY |
| | YPTTTSSGTYGAVAISPGYTATQSSIAWLGPRLASHGFVVITID |
| | TQTTFDQPDSRARQLMAALNYLVNRSSVRSRIDSSRLAVMGH |
| | SMGGGGTLRAAEDNPSLKAAIPLTPWHTNKNWSSVRVPTLII |
| | GAENDTIAPVSSHAKPFYNSLPSSTPKAYLELNGASHFAPNSS |
| | NTTIGKYSVSWLKRFVDNDTRYSQFLCPAPHDDSAISEYRSTC |
| | PY |
| 11 | AANPYERGPDPTEASLEASSGPFSVSETSVSRLSASGFGGGTIY |
| | YPTTTSSGTYGAVAISPGYTATQSSIAWLGPRLASHGFVVITID |
| | TNTTFDQPDSRARQLMAALNYLVNRSSVRSRIDSSRLAVMGH |
| | SMGGGGTLRAAEDNPSLKAAIPLTPWHTNKDWSSVRVPTLII |
| | GAENDTIAPVSSHAKPFYNSLPSSTPKAYLELNGASHFAPNSS |
| | NTTIGKYSVSWLKRFVDNDTRYSQFLCPAPHDDSAISEYRSTC |
| | PY |
| 12 | AANPYERGPDPTEASLEASSGPFSVSETSVSRLSASGFGGGTIY |
| | YPTTTSSGTYGAVAISPGYTATQSSIAWLGPRLASHGFVVITID |
| | TNTTFDQPDSRARQLMAALNYLVNRSSVRSRIDSSRLAVMGH |
| | SMGGGGTLRAAEDNPSLKAAIPLTPWHTNKNWSSVRVPTLII |
| | GAENDTIAPVSSHAKPFYNSLPASTPKAYLELNGASHFAPNSS |
| | NTTIGKYSVSWLKRFVDNDTRYSQFLCPAPHDDSAISEYRSTC |
| | PY |
| 13 | AANPYERGPDPTEASLEASSGPFSVSETSVSRLSASGFGGGTIY |
| | YPTTTSSGTYGAVAISPGYTATQSSIAWLGPRLASHGFVVITID |
| | TNTTFDQPDSRARQLMAALNYLVNRSSVRSRIDSSRLAVMGH |
| | SMGGGGTLRAAEDNPSLKAAIPLTPWHTNKNWSSVRVPTLII |
| | GAENDTIAPVSSHAKPFYNSLPSSTPKAYLELNGASHFAPNSS |
| | NTTIGKYSVSWLKRFVDNDTRYNQFLCPAPHDDSAISEYRSTC |
| | PY |
| 14 | AANPYERGPDPTDSSLEASSGPFSVSETSVSRLSASGFGGGTIY |
| | YPTTTSEGTYGAVAISPGYTATQSSIAWLGPRLASHGFVVITID |
| | TNTTFDQPDSRARQLMAALNYLVNRSSVRSRIDSSRLAVMGH |
| | SMGGGGTLRAAEDNPSLKAAIPLTPWHTNKNWSSVRVPTLII |
| | GAENDTIAPVSSHAKPFYNSLPSSTPKAYLELNGASHFAPNSS |
| | NTTIGKYSIAWLKRFVDNDTRYSQFLCPAPHDDSAISEYRSTC |
| | PY |
| 15 | AANPYERGPDPTESSLEASSGPFSVSQTSVSRLSASGFGGGTIY |
| | YPTTTSEGTYGAVAISPGYTATQSSIAWLGPRLASHGFVVITID |
| | TNTTFDQPDSRARQLMAALNYLVNRSSVRSRIDSSRLAVMGH |
| | SMGGGGTLRAAEDNPSLKAAIPLTPWHTNKNWSSVRVPTLII |
| | GAENDTIAPVSSHAKPFYNSLPSSTPKAYLELNGASHFAPNSS |
| | NTTIGKYSIAWLKRFVDNDTRYSQFLCPAPHDDSAISEYRSTC |
| | PY |
| 16 | AANPYERGPDPTESSLEASSGPFSVSETSVSRLSVSGFGGGTIY |
| | YPTTTSEGTYGAVAISPGYTATQSSIAWLGPRLASHGFVVITID |
| | TNTTFDQPDSRARQLMAALNYLVNRSSVRSRIDSSRLAVMGH |
| | SMGGGGTLRAAEDNPSLKAAIPLTPWHTNKNWSSVRVPTLII |
| | GAENDTIAPVSSHAKPFYNSLPSSTPKAYLELNGASHFAPNSS |
| | NTTIGKYSIAWLKRFVDNDTRYSQFLCPAPHDDSAISEYRSTC |
| | PY |
| 17 | AANPYERGPDPTESSLEASSGPFSVSETSVSRLSASGFGGGTIY |
| | YPTSTSEGTYGAVAISPGYTATQSSIAWLGPRLASHGFVVITID |
| | TNTTFDQPDSRARQLMAALNYLVNRSSVRSRIDSSRLAVMGH |
| | SMGGGGTLRAAEDNPSLKAAIPLTPWHTNKNWSSVRVPTLII |
| | GAENDTIAPVSSHAKPFYNSLPSSTPKAYLELNGASHFAPNSS |
| | NTTIGKYSIAWLKRFVDNDTRYSQFLCPAPHDDSAISEYRSTC |
| | PY |
| 18 | AANPYERGPDPTESSLEASSGPFSVSETSVSRLSASGFGGGTIY |
| | YPTTTSSGTYGAVAISPGYTATQSSIAWLGPRLASHGFVVITID |
| | TNTTFDQPDSRARQLMAALNYLVNRSSVRSRIDSSRLAVMGH |
| | SMGGGGTLRAAEDNPSLKAAIPLTPWHTNKNWSSVRVPTLII |
| | GAENDTIAPVSSHAKPFYNSLPSSTPKAYLELNGASHFAPNSS |
| | NTTIGKYSIAWLKRFVDNDTRYSQFLCPAPHDDSAISEYRSTC |
| | PY |
| 19 | AANPYERGPDPTESSLEASSGPFSVSETSVSRLSASGFGGGTIY |
| | YPTTTSEGTFGAVAISPGYTATQSSIAWLGPRLASHGFVVITID |
| | TNTTFDQPDSRARQLMAALNYLVNRSSVRSRIDSSRLAVMGH |
| | SMGGGGTLRAAEDNPSLKAAIPLTPWHTNKNWSSVRVPTLII |
| | GAENDTIAPVSSHAKPFYNSLPSSTPKAYLELNGASHFAPNSS |
| | NTTIGKYSIAWLKRFVDNDTRYSQFLCPAPHDDSAISEYRSTC |
| | PY |
| 20 | AANPYERGPDPTESSLEASSGPFSVSETSVSRLSASGFGGGTIY |
| | YPTTTSEGTYGAVAISPGYTATQSSIAWLGRRLASHGFVVITID |
| | TNTTFDQPDSRARQLMAALNYLVNRSSVRSRIDSSRLAVMGH |
| | SMGGGGTLRAAEDNPSLKAAIPLTPWHTNKNWSSVRVPTLII |
| | GAENDTIAPVSSHAKPFYNSLPSSTPKAYLELNGASHFAPNSS |
| | NTTIGKYSIAWLKRFVDNDTRYSQFLCPAPHDDSAISEYRSTC |
| | PY |
| 21 | AANPYERGPDPTESSLEASSGPFSVSETSVSRLSASGFGGGTIY |
| | YPTTTSEGTYGAVAISPGYTATQSSIAWLGPRLASHGFVVITID |
| | TNTTLDQPDSRARQLMAALNYLVNRSSVRSRIDSSRLAVMGH |
| | SMGGGGTLRAAEDNPSLKAAIPLTPWHTNKNWSSVRVPTLII |
| | GAENDTIAPVSSHAKPFYNSLPSSTPKAYLELNGASHFAPNSS |
| | NTTIGKYSIAWLKRFVDNDTRYSQFLCPAPHDDSAISEYRSTC |
| | PY |
| 22 | AANPYERGPDPTESSLEASSGPFSVSETSVSRLSASGFGGGTIY |
| | YPTTTSEGTYGAVAISPGYTATQSSIAWLGPRLASHGFVVITID |
| | TNTTFDQPDSRARQLMAALDYLVNRSSVRSRIDSSRLAVMGH |
| | SMGGGGTLRAAEDNPSLKAAIPLTPWHTNKNWSSVRVPTLII |
| | GAENDTIAPVSSHAKPFYNSLPSSTPKAYLELNGASHFAPNSS |
| | NTTIGKYSIAWLKRFVDNDTRYSQFLCPAPHDDSAISEYRSTC |
| | PY |
| 23 | AANPYERGPDPTESSLEASSGPFSVSETSVSRLSASGFGGGTIY |
| | YPTTTSEGTYGAVAISPGYTATQSSIAWLGPRLASHGFVVITID |
| | TNTTFDQPDSRARQLMAALNYLVNRSSVRSRIDPSRLAVMGH |
| | SMGGGGTLRAAEDNPSLKAAIPLTPWHTNKNWSSVRVPTLII |
| | GAENDTIAPVSSHAKPFYNSLPSSTPKAYLELNGASHFAPNSS |
| | NTTIGKYSIAWLKRFVDNDTRYSQFLCPAPHDDSAISEYRSTC |
| | PY |
| 24 | AANPYERGPDPTESSLEASSGPFSVSETSVSRLSASGFGGGTIY |
| | YPTTTSEGTYGAVAISPGYTATQSSIAWLGPRLASHGFVVITID |
| | TNTTFDQPDSRARQLMAALNYLVNRSSVRSRIDSSRLAVMGH |
| | SMGGGGTLRAAEDNPSLKAAIPLTPWHTNKNWSSVRVPTLII |
| | GAENDTIAPVSSHAKPFYNSLPSSTPKAYLELNGASHFAPNTS |
| | NTTIGKYSIAWLKRFVDNDTRYSQFLCPAPHDDSAISEYRSTC |
| | PY |
| 25 | AANPYERGPDPTESSLEASSGPFSVSETSVSRLSASGFGGGTIY |
| | YPTTTSEGTYGAVAISPGYTATQSSIAWLGPRLASHGFVVITID |
| | TNTTFDQPDSRARQLMAALNYLVNRSSVRSRIDSSRLAVMGH |
| | SMGGGGTLRAAEDNPSLKAAIPLTPWHTNKNWSSVRVPTLII |
| | GAENDTIAPVSSHAKPFYNSLPSSTPKAYLELNGASHFAPNSS |
| | NTTIGKYSISWLKRFVDNDTRYSQFLCPAPHDDSAISEYRSTCP |
| | Y |
| 26 | AANPYERGPDPTESSLEASSGPFSVSETSVSRLSASGFGGGTIY |
| | YPTTTSEGTYGAVAISPGYTATQSSIAWLGPRLASHGFVVITID |
| | TNTTFDQPDSRARQLMAALNYLVNRSSVRSRIDSSRLAVMGH |
| | SMGGGGTLRAAEDNPSLKAAIPLTPWHTNKNWSSVRVPTLII |
| | GAENDTIAPVSSHAKPFYNSLPSSTPKAYLELNGASHFAPNSS |
| | NTTIGKYSIAWLKRFVDNDTRYTQFLCPAPHDDSAISEYRSTC |
| | PY |
| 27 | AANPYERGPDPTESSLEASSGPFSVSETSVSRLSASGFGGGTIY |
| | YPTTTSEGTYGAVAISPGYTATQSSIAWLGPRLASHGFVVITID |
| | TNTTFDQPDSRARQLMAALNYLVNRSSVRSRIDSSRLAVMGH |
| | SMGGGGTLRAAEDNPSLKAAIPLTPWHTNKNWSSVRVPTLII |
| | GAENDTIAPVSSHAKPFYNSLPSSTPKAYLELNGASHFAPNSS |
| | NTTIGKYSIAWLKRFVDNDTRYSQFLCPAPSDDSAISEYRSTCP |
| | Y |
| 28 | AANPYERGPDPTESSLEASSGPFSVSETSVSRLSASGFGGGTIY |
| | YPTTTSEGTYGAVAISPGYTATQSSIAWLGPRLASHGFVVITID |
| | TNTTFDQPDSRARQLMAALNYLVNRSSVRSRIDSSRLAVMGH |
| | SMGGGGTLRAAEDNPSLKAAIPLTPWHTNKNWSSVRVPTLII |
| | GAENDTIAPVSSHAKPFYNSLPSSTPKAYLELNGASHFAPNSS |
| | NTTIGKYSIAWLKRFVDNDTRYSQFLCPAPHDDAAISEYRSTC |
| | PY |
| 29 | AANPYERGPDPTEASLEASSGPFSVSETSVSRLSASGFGGGTIY |
| | YPTTTSSGTYGAVAISPGYTATQSSIAWLGPRLASHGFVVITID |
| | TNTTFDQPDSRARQLMAALNYLVNRSSVRSRIDSSRLAVMGH |
| | SMGGGGTLRAAEDNPSLKAAIPLTPWHTNKNWSSVRVPTLII |
| | GAENDTIAPVSSHAKPFYNSLPSSTPKAYLELCGASHVAPNSS |
| | NTTIGKYSVSWLKRFVDNDTRYSQFLCPAPHDDSAICEYRSTC |
| | PY |
| 30 | AANP (an N-terminal cellular export signal |
| | peptide) |
| 31 | GGGSTPLPLPQQQPPQQEPPPPPVPLASRAACEALKDGNGDM |
| | VWPNAATVVEVAAWRDAAPATASAAALPEHCEVSGAIAKRT |
| | GIDGYPYEIKFRLRMPAEWNGRFFMEGGSGTNGSLSAATGSIG |
| | GGQIASALSRNFATIATDGGHDNAVNDNPDALGTVAFGLDPQ |
| | ARLDMGYNSYDQVTQAGKAAVARFYGRAADKSYFIGCSEGG |
| | REGMMLSQRFPSHYDGIVAGAPGYQLPKAGISGAWTTQSLAP |
| | AAVGLDAQGVPLINKSFSDADLHLLSQAILGTCDALDGLADGI |
| | VDNYRACQAAFDPATAANPANGQALQCVGAKTADCLSPVQ |
| | VTAIKRAMAGPVNSAGTPLYNRWAWDAGMSGLSGTTYNQG |
| | WRSWWLGSFNSSANNAQRVSGFSARSWLVDFATPPEPMPMT |
| | QVAARMMKFDFDIDPLKIWATSGQFTQSSMDWHGATSTDLA |
| | AFRDRGGKMILYHGMSDAAFSALDTADYYERLGAAMPGAA |
| | GFARLFLVPGMNHCSGGPGTDRFDMLTPLVAWVERGEAPDQ |
| | ISAWSGTPGYFGVAARTRPLCPYPQIARYKGSGDINTEANFAC |
| | AAPP |
| 32 | PATASAAALPEHCEVSGAIAKRTGIDGYPYEIKFRLRMPAEWN |
| | GRFFMEGGSGTNGSLSA |
| | ATGSIGGGQIASALSRGFAVIATDGGHDNAVNDNPDALGTVA |
| | FGLDPQARLDMGYNSYDQ |
| | VTQAGKAAVARFYGRAADKSYFIGCSEGGREGMMLSQRFPS |
| | HYDGIVAGAPGYQLPKAGI |
| | SGAWTTQSLAPAAVGLDAQGVPLINKSFSDADLHLLSQAILG |
| | TCDALDGLADGIVDNYRA |
| | CQAAFDPATAANPANGQALQCVGAKTADCLSPVQVTAIKRA |
| | MAGPVNSAGTPLYNRWAWD |
| | AGMSGLSGTTYNQGWRSWWLGSFNSSANNAQRVSGFSARS |
| | WLVDFATPPEPMPMTQVAAR |
| | MMKFDFDIDPLKIWATSGQFTQSSMDWHGATSTDLAAFRDR |
| | GGKMILYHGMSDAAFSALD |
| | TADYYERLGAAMPGAAGFARLFLVPGMNHCSGGPGTDRFD |
| | MLTPLVAWVERGEAPDQISA |
| | WSGTPGYFGVAARTRPLCPYPQIARYKGSGDINTEANFACAA |
| | PP |
| 33 | GGGSTPLPLPQQQPPQQEPPPPPVPLASRAACEALKDGNGDM |
| | VWPNAATVVEVAAWRDAA |
| | PATASAAALPEHCEVSGAIAKRTGIDGYPYEIKFRLRMPAEWN |
| | GRFFMEGGSGTNGSLSA |
| | ATGSIGGGQIASALSRGFAVIATDGGHDNAVNDNPDALGTVA |
| | FGLDPQARLDMGVNSYDQ |
| | VTQAGKAAVARFYGRAADKSYFIGCSEGGREGMMLSQRFPS |
| | HYDGIVAGAPGYQLPKAGI |
| | SGAWTTQSLAPAAVGLDAQGVPLINKSFSDADLHLLSQAILG |
| | TCDALDGLADGIVDNYRA |
| | CQAAFDPATAANPANGQALQCVGAKTADCLSPVQVTAIKRA |
| | MAGPVNSAGTPLYNRWAWD |
| | AGMSGLSGTTYNQGWRSWWLGSFNSSANNAQRVSGFSARS |
| | WLVDFATPPEPMPMTQVAAR |
| | MMKFDFDIDPLKIWATSGQFTQSSMDWHGATSTDLAAFRDR |
| | GGKMILYHGMSDAAFSALD |
| | TADYYERLGAAMPGAAGFARLFLVPGMNHCSGGPGTDRFD |
| | MLTPLVAWVERGEAPDQISA |
| | WSGTPGYFGVAARTRPLCPYPQIARYKGSGDINTEANFACAA |
| | PP |
| 34 | GGGSTPLPLPQQQPPQQEPPPPPVPLASRAACEALKDGNGDM |
| | VWPNAAVVVEVAAWRDAA |
| | PATASAAALPEHCEVSGAIAKRTGIDGYPYEIKFRLRMPAEWN |
| | GRFFMEGGSGTNGSLSA |
| | ATGSIGGGQIASALSRGFAVIATDGGHDNAVNDNPDALGTVA |
| | FGLDPQARLDMGVNSYDQ |
| | VTQAGKAAVARFYGRAADKSYFIGCSEGGREGMMLSQRFPS |
| | HYDGIVAGAPGYQLPKAGI |
| | SGAWTTQSLAPAAVGLDAQGVPLINKSFSDADLHLLSQAILG |
| | TCDALDGLADGIVDNYRA |
| | CQAAFDPATAANPANGQALQCVGAKTADCLSPVQVTAIKRA |
| | MAGPVNSAGTPLYNRWAWD |
| | AGMSGLSGTTYNQGWRSWWLGSFNSSANNAQRVSGFSARS |
| | WLVDFATPPEPMPMTQVAAR |
| | MMKFDFDIDPLKIWATSGQFTQSSMDWHGATSTDLAAFRDR |
| | GGKMILYHGMSDAAFSALD |
| | TADYYERLGAAMPGAAGFARLFLVPGMNHCSGGPGTDRFD |
| | MLTPLVAWVERGEAPDQISA |
| | WSGTPGYFGVAARTRPLCPYPQIARYKGSGDINTEANFACAA |
| | PP |
| 35 | GGGSTPLPLPQQQPPQQEPPPPPVPLASRAACEALKDGNGDM |
| | VWPNAATVVEVAAWRDPA |
| | PATASAAALPEHCEVSGAIAKRTGIDGYPYEIKFRLRMPAEWN |
| | GRFFMEGGSGTNGSLSA |
| | ATGSIGGGQIASALSRGFAVIATDGGHDNAVNDNPDALGTVA |
| | FGLDPQARLDMGVNSYDQ |
| | VTQAGKAAVARFYGRAADKSYFIGCSEGGREGMMLSQRFPS |
| | HYDGIVAGAPGYQLPKAGI |
| | SGAWTTQSLAPAAVGLDAQGVPLINKSFSDADLHLLSQAILG |
| | TCDALDGLADGIVDNYRA |
| | CQAAFDPATAANPANGQALQCVGAKTADCLSPVQVTAIKRA |
| | MAGPVNSAGTPLYNRWAWD |
| | AGMSGLSGTTYNQGWRSWWLGSFNSSANNAQRVSGFSARS |
| | WLVDFATPPEPMPMTQVAAR |
| | MMKFDFDIDPLKIWATSGQFTQSSMDWHGATSTDLAAFRDR |
| | GGKMILYHGMSDAAFSALD |
| | TADYYERLGAAMPGAAGFARLFLVPGMNHCSGGPGTDRFD |
| | MLTPLVAWVERGEAPDQISA |
| | WSGTPGYFGVAARTRPLCPYPQIARYKGSGDINTEANFACAA |
| | PP |
| 36 | GGGSTPLPLPQQQPPQQEPPPPPVPLASRAACEALKDGNGDM |
| | VWPNAATVVEVAAWRDAA |
| | PATASAAALPEHCEVSGAIAKRTGIDGYPYAIKFRLRMPAEW |
| | NGRFFMEGGSGTNGSLSA |
| | ATGSIGGGQIASALSRGFAVIATDGGHDNAVNDNPDALGTVA |
| | FGLDPQARLDMGVNSYDQ |
| | VTQAGKAAVARFYGRAADKSYFIGCSEGGREGMMLSQRFPS |
| | HYDGIVAGAPGYQLPKAGI |
| | SGAWTTQSLAPAAVGLDAQGVPLINKSFSDADLHLLSQAILG |
| | TCDALDGLADGIVDNYRA |
| | CQAAFDPATAANPANGQALQCVGAKTADCLSPVQVTAIKRA |
| | MAGPVNSAGTPLYNRWAWD |
| | AGMSGLSGTTYNQGWRSWWLGSFNSSANNAQRVSGFSARS |
| | WLVDFATPPEPMPMTQVAAR |
| | MMKFDFDIDPLKIWATSGQFTQSSMDWHGATSTDLAAFRDR |
| | GGKMILYHGMSDAAFSALD |
| | TADYYERLGAAMPGAAGFARLFLVPGMNHCSGGPGTDRFD |
| | MLTPLVAWVERGEAPDQISA |
| | WSGTPGYFGVAARTRPLCPYPQIARYKGSGDINTEANFACAA |
| | PP |
| 37 | GGGSTPLPLPQQQPPQQEPPPPPVPLASRAACEALKDGNGDM |
| | VWPNAATVVEVAAWRDAA |
| | PATASAAALPEHCEVSGAIAKRTGIDGYPYEIKFRLRLPAEWN |
| | GRFFMEGGSGTNGSLSA |
| | ATGSIGGGQIASALSRGFAVIATDGGHDNAVNDNPDALGTVA |
| | FGLDPQARLDMGVNSYDQ |
| | VTQAGKAAVARFYGRAADKSYFIGCSEGGREGMMLSQRFPS |
| | HYDGIVAGAPGYQLPKAGI |
| | SGAWTTQSLAPAAVGLDAQGVPLINKSFSDADLHLLSQAILG |
| | TCDALDGLADGIVDNYRA |
| | CQAAFDPATAANPANGQALQCVGAKTADCLSPVQVTAIKRA |
| | MAGPVNSAGTPLYNRWAWD |
| | AGMSGLSGTTYNQGWRSWWLGSFNSSANNAQRVSGFSARS |
| | WLVDFATPPEPMPMTQVAAR |
| | MMKFDFDIDPLKIWATSGQFTQSSMDWHGATSTDLAAFRDR |
| | GGKMILYHGMSDAAFSALD |
| | TADYYERLGAAMPGAAGFARLFLVPGMNHCSGGPGTDRFD |
| | MLTPLVAWVERGEAPDQISA |
| | WSGTPGYFGVAARTRPLCPYPQIARYKGSGDINTEANFACAA |
| | PP |
| 38 | GGGSTPLPLPQQQPPQQEPPPPPVPLASRAACEALKDGNGDM |
| | VWPNAATVVEVAAWRDAA |
| | PATASAAALPEHCEVSGAIAKRTGIDGYPYEIKFRLRMPAEWN |
| | GRFFMQGGSGTNGSLSA |
| | ATGSIGGGQIASALSRGFAVIATDGGHDNAVNDNPDALGTVA |
| | FGLDPQARLDMGVNSYDQ |
| | VTQAGKAAVARFYGRAADKSYFIGCSEGGREGMMLSQRFPS |
| | HYDGIVAGAPGYQLPKAGI |
| | SGAWTTQSLAPAAVGLDAQGVPLINKSFSDADLHLLSQAILG |
| | TCDALDGLADGIVDNYRA |
| | CQAAFDPATAANPANGQALQCVGAKTADCLSPVQVTAIKRA |
| | MAGPVNSAGTPLYNRWAWD |
| | AGMSGLSGTTYNQGWRSWWLGSFNSSANNAQRVSGFSARS |
| | WLVDFATPPEPMPMTQVAAR |
| | MMKFDFDIDPLKIWATSGQFTQSSMDWHGATSTDLAAFRDR |
| | GGKMILYHGMSDAAFSALD |
| | TADYYERLGAAMPGAAGFARLFLVPGMNHCSGGPGTDRFD |
| | MLTPLVAWVERGEAPDQISA |
| | WSGTPGYFGVAARTRPLCPYPQIARYKGSGDINTEANFACAA |
| | PP |
| 39 | GGGSTPLPLPQQQPPQQEPPPPPVPLASRAACEALKDGNGDM |
| | VWPNAATVVEVAAWRDAA |
| | PATASAAALPEHCEVSGAIAKRTGIDGYPYEIKFRLRMPAEWN |
| | GRFFMEGGGGTNGSLSA |
| | ATGSIGGGQIASALSRGFAVIATDGGHDNAVNDNPDALGTVA |
| | FGLDPQARLDMGVNSYDQ |
| | VTQAGKAAVARFYGRAADKSYFIGCSEGGREGMMLSQRFPS |
| | HYDGIVAGAPGYQLPKAGI |
| | SGAWTTQSLAPAAVGLDAQGVPLINKSFSDADLHLLSQAILG |
| | TCDALDGLADGIVDNYRA |
| | CQAAFDPATAANPANGQALQCVGAKTADCLSPVQVTAIKRA |
| | MAGPVNSAGTPLYNRWAWD |
| | AGMSGLSGTTYNQGWRSWWLGSFNSSANNAQRVSGFSARS |
| | WLVDFATPPEPMPMTQVAAR |
| | MMKFDFDIDPLKIWATSGQFTQSSMDWHGATSTDLAAFRDR |
| | GGKMILYHGMSDAAFSALD |
| | TADYYERLGAAMPGAAGFARLFLVPGMNHCSGGPGTDRFD |
| | MLTPLVAWVERGEAPDQISA |
| | WSGTPGYFGVAARTRPLCPYPQIARYKGSGDINTEANFACAA |
| | PP |
| 40 | GGGSTPLPLPQQQPPQQEPPPPPVPLASRAACEALKDGNGDM |
| | VWPNAATVVEVAAWRDAA |
| | PATASAAALPEHCEVSGAIAKRTGIDGYPYEIKFRLRMPAEWN |
| | GRFFMEGGSGTDGSLSA |
| | ATGSIGGGQIASALSRGFAVIATDGGHDNAVNDNPDALGTVA |
| | FGLDPQARLDMGVNSYDQ |
| | VTQAGKAAVARFYGRAADKSYFIGCSEGGREGMMLSQRFPS |
| | HYDGIVAGAPGYQLPKAGI |
| | SGAWTTQSLAPAAVGLDAQGVPLINKSFSDADLHLLSQAILG |
| | TCDALDGLADGIVDNYRA |
| | CQAAFDPATAANPANGQALQCVGAKTADCLSPVQVTAIKRA |
| | MAGPVNSAGTPLYNRWAWD |
| | AGMSGLSGTTYNQGWRSWWLGSFNSSANNAQRVSGFSARS |
| | WLVDFATPPEPMPMTQVAAR |
| | MMKFDFDIDPLKIWATSGQFTQSSMDWHGATSTDLAAFRDR |
| | GGKMILYHGMSDAAFSALD |
| | TADYYERLGAAMPGAAGFARLFLVPGMNHCSGGPGTDRFD |
| | MLTPLVAWVERGEAPDQISA |
| | WSGTPGYFGVAARTRPLCPYPQIARYKGSGDINTEANFACAA |
| | PP |
| 41 | GGGSTPLPLPQQQPPQQEPPPPPVPLASRAACEALKDGNGDM |
| | VWPNAATVVEVAAWRDAA |
| | PATASAAALPEHCEVSGAIAKRTGIDGYPYEIKFRLRMPAEWN |
| | GRFFMEGGSGTNGSLSA |
| | ATGSIGGGQIASALSRNFAVIATDGGHDNAVNDNPDALGTVA |
| | FGLDPQARLDMGVNSYDQ |
| | VTQAGKAAVARFYGRAADKSYFIGCSEGGREGMMLSQRFPS |
| | HYDGIVAGAPGYQLPKAGI |
| | SGAWTTQSLAPAAVGLDAQGVPLINKSFSDADLHLLSQAILG |
| | TCDALDGLADGIVDNYRA |
| | CQAAFDPATAANPANGQALQCVGAKTADCLSPVQVTAIKRA |
| | MAGPVNSAGTPLYNRWAWD |
| | AGMSGLSGTTYNQGWRSWWLGSFNSSANNAQRVSGFSARS |
| | WLVDFATPPEPMPMTQVAAR |
| | MMKFDFDIDPLKIWATSGQFTQSSMDWHGATSTDLAAFRDR |
| | GGKMILYHGMSDAAFSALD |
| | TADYYERLGAAMPGAAGFARLFLVPGMNHCSGGPGTDRFD |
| | MLTPLVAWVERGEAPDQISA |
| | WSGTPGYFGVAARTRPLCPYPQIARYKGSGDINTEANFACAA |
| | PP |
| 42 | GGGSTPLPLPQQQPPQQEPPPPPVPLASRAACEALKDGNGDM |
| | VWPNAATVVEVAAWRDAA |
| | PATASAAALPEHCEVSGAIAKRTGIDGYPYEIKFRLRMPAEWN |
| | GRFFMEGGSGTNGSLSA |
| | ATGSIGGGQIASALSRGFAVIGTDGGHDNAVNDNPDALGTVA |
| | FGLDPQARLDMGVNSYDQ |
| | VTQAGKAAVARFYGRAADKSYFIGCSEGGREGMMLSQRFPS |
| | HYDGIVAGAPGYQLPKAGI |
| | SGAWTTQSLAPAAVGLDAQGVPLINKSFSDADLHLLSQAILG |
| | TCDALDGLADGIVDNYRA |
| | CQAAFDPATAANPANGQALQCVGAKTADCLSPVQVTAIKRA |
| | MAGPVNSAGTPLYNRWAWD |
| | AGMSGLSGTTYNQGWRSWWLGSFNSSANNAQRVSGFSARS |
| | WLVDFATPPEPMPMTQVAAR |
| | MMKFDFDIDPLKIWATSGQFTQSSMDWHGATSTDLAAFRDR |
| | GGKMILYHGMSDAAFSALD |
| | TADYYERLGAAMPGAAGFARLFLVPGMNHCSGGPGTDRFD |
| | MLTPLVAWVERGEAPDQISA |
| | WSGTPGYFGVAARTRPLCPYPQIARYKGSGDINTEANFACAA |
| | PP |
| 43 | GGGSTPLPLPQQQPPQQEPPPPPVPLASRAACEALKDGNGDM |
| | VWPNAATVVEVAAWRDAA |
| | PATASAAALPEHCEVSGAIAKRTGIDGYPYEIKFRLRMPAEWN |
| | GRFFMEGGSGTNGSLSA |
| | ATGSIGGGQIASALSRGFAVIATDGGHDNAVNDNPDALGTVA |
| | FGLDPQARLLMGVNSYDQ |
| | VTQAGKAAVARFYGRAADKSYFIGCSEGGREGMMLSQRFPS |
| | HYDGIVAGAPGYQLPKAGI |
| | SGAWTTQSLAPAAVGLDAQGVPLINKSFSDADLHLLSQAILG |
| | TCDALDGLADGIVDNYRA |
| | CQAAFDPATAANPANGQALQCVGAKTADCLSPVQVTAIKRA |
| | MAGPVNSAGTPLYNRWAWD |
| | AGMSGLSGTTYNQGWRSWWLGSFNSSANNAQRVSGFSARS |
| | WLVDFATPPEPMPMTQVAAR |
| | MMKFDFDIDPLKIWATSGQFTQSSMDWHGATSTDLAAFRDR |
| | GGKMILYHGMSDAAFSALD |
| | TADYYERLGAAMPGAAGFARLFLVPGMNHCSGGPGTDRFD |
| | MLTPLVAWVERGEAPDQISA |
| | WSGTPGYFGVAARTRPLCPYPQIARYKGSGDINTEANFACAA |
| | PP |
| 44 | GGGSTPLPLPQQQPPQQEPPPPPVPLASRAACEALKDGNGDM |
| | VWPNAATVVEVAAWRDAA |
| | PATASAAALPEHCEVSGAIAKRTGIDGYPYEIKFRLRMPAEWN |
| | GRFFMEGGSGTNGSLSA |
| | ATGSIGGGQIASALSRGFAVIATDGGHDNAVNDNPDALGTVA |
| | FGLDPQARLDYGVNSYDQ |
| | VTQAGKAAVARFYGRAADKSYFIGCSEGGREGMMLSQRFPS |
| | HYDGIVAGAPGYQLPKAGI |
| | SGAWTTQSLAPAAVGLDAQGVPLINKSFSDADLHLLSQAILG |
| | TCDALDGLADGIVDNYRA |
| | CQAAFDPATAANPANGQALQCVGAKTADCLSPVQVTAIKRA |
| | MAGPVNSAGTPLYNRWAWD |
| | AGMSGLSGTTYNQGWRSWWLGSFNSSANNAQRVSGFSARS |
| | WLVDFATPPEPMPMTQVAAR |
| | MMKFDFDIDPLKIWATSGQFTQSSMDWHGATSTDLAAFRDR |
| | GGKMILYHGMSDAAFSALD |
| | TADYYERLGAAMPGAAGFARLFLVPGMNHCSGGPGTDRFD |
| | MLTPLVAWVERGEAPDQISA |
| | WSGTPGYFGVAARTRPLCPYPQIARYKGSGDINTEANFACAA |
| | PP |
| 45 | GGGSTPLPLPQQQPPQQEPPPPPVPLASRAACEALKDGNGDM |
| | VWPNAATVVEVAAWRDAA |
| | PATASAAALPEHCEVSGAIAKRTGIDGYPYEIKFRLRMPAEWN |
| | GRFFMEGGSGTNGSLSA |
| | ATGSIGGGQIASALSRGFAVIATDGGHDNAVNDNPDALGTVA |
| | FGLDPQARLDMGVNAYDQ |
| | VTQAGKAAVARFYGRAADKSYFIGCSEGGREGMMLSQRFPS |
| | HYDGIVAGAPGYQLPKAGI |
| | SGAWTTQSLAPAAVGLDAQGVPLINKSFSDADLHLLSQAILG |
| | TCDALDGLADGIVDNYRA |
| | CQAAFDPATAANPANGQALQCVGAKTADCLSPVQVTAIKRA |
| | MAGPVNSAGTPLYNRWAWD |
| | AGMSGLSGTTYNQGWRSWWLGSFNSSANNAQRVSGFSARS |
| | WLVDFATPPEPMPMTQVAAR |
| | MMKFDFDIDPLKIWATSGQFTQSSMDWHGATSTDLAAFRDR |
| | GGKMILYHGMSDAAFSALD |
| | TADYYERLGAAMPGAAGFARLFLVPGMNHCSGGPGTDRFD |
| | MLTPLVAWVERGEAPDQISA |
| | WSGTPGYFGVAARTRPLCPYPQIARYKGSGDINTEANFACAA |
| | PP |
| 46 | GGGSTPLPLPQQQPPQQEPPPPPVPLASRAACEALKDGNGDM |
| | VWPNAATVVEVAAWRDAA |
| | PATASAAALPEHCEVSGAIAKRTGIDGYPYEIKFRLRMPAEWN |
| | GRFFMEGGSGTNGSLSA |
| | ATGSIGGGQIASALSRGFAVIATDGGHDNAVNDNPDALGTVA |
| | FGLDPQARLDMGVNSYDQ |
| | VTQAAKAAVARFYGRAADKSYFIGCSEGGREGMMLSQRFPS |
| | HYDGIVAGAPGYQLPKAGI |
| | SGAWTTQSLAPAAVGLDAQGVPLINKSFSDADLHLLSQAILG |
| | TCDALDGLADGIVDNYRA |
| | CQAAFDPATAANPANGQALQCVGAKTADCLSPVQVTAIKRA |
| | MAGPVNSAGTPLYNRWAWD |
| | AGMSGLSGTTYNQGWRSWWLGSFNSSANNAQRVSGFSARS |
| | WLVDFATPPEPMPMTQVAAR |
| | MMKFDFDIDPLKIWATSGQFTQSSMDWHGATSTDLAAFRDR |
| | GGKMILYHGMSDAAFSALD |
| | TADYYERLGAAMPGAAGFARLFLVPGMNHCSGGPGTDRFD |
| | MLTPLVAWVERGEAPDQISA |
| | WSGTPGYFGVAARTRPLCPYPQIARYKGSGDINTEANFACAA |
| | PP |
| 47 | GGGSTPLPLPQQQPPQQEPPPPPVPLASRAACEALKDGNGDM |
| | VWPNAATVVEVAAWRDAA |
| | PATASAAALPEHCEVSGAIAKRTGIDGYPYEIKFRLRMPAEWN |
| | GRFFMEGGSGTNGSLSA |
| | ATGSIGGGQIASALSRGFAVIATDGGHDNAVNDNPDALGTVA |
| | FGLDPQARLDMGVNSYDQ |
| | VTQAGKALVARFYGRAADKSYFIGCSEGGREGMMLSQRFPS |
| | HYDGIVAGAPGYQLPKAGI |
| | SGAWTTQSLAPAAVGLDAQGVPLINKSFSDADLHLLSQAILG |
| | TCDALDGLADGIVDNYRA |
| | CQAAFDPATAANPANGQALQCVGAKTADCLSPVQVTAIKRA |
| | MAGPVNSAGTPLYNRWAWD |
| | AGMSGLSGTTYNQGWRSWWLGSFNSSANNAQRVSGFSARS |
| | WLVDFATPPEPMPMTQVAAR |
| | MMKFDFDIDPLKIWATSGQFTQSSMDWHGATSTDLAAFRDR |
| | GGKMILYHGMSDAAFSALD |
| | TADYYERLGAAMPGAAGFARLFLVPGMNHCSGGPGTDRFD |
| | MLTPLVAWVERGEAPDQISA |
| | WSGTPGYFGVAARTRPLCPYPQIARYKGSGDINTEANFACAA |
| | PP |
| 48 | GGGSTPLPLPQQQPPQQEPPPPPVPLASRAACEALKDGNGDM |
| | VWPNAATVVEVAAWRDAA |
| | PATASAAALPEHCEVSGAIAKRTGIDGYPYEIKFRLRMPAEWN |
| | GRFFMEGGSGTNGSLSA |
| | ATGSIGGGQIASALSRGFAVIATDGGHDNAVNDNPDALGTVA |
| | FGLDPQARLDMGVNSYDQ |
| | VTQAGKAAVARFYGRAPDKSYFIGCSEGGREGMMLSQRFPS |
| | HYDGIVAGAPGYQLPKAGI |
| | SGAWTTQSLAPAAVGLDAQGVPLINKSFSDADLHLLSQAILG |
| | TCDALDGLADGIVDNYRA |
| | CQAAFDPATAANPANGQALQCVGAKTADCLSPVQVTAIKRA |
| | MAGPVNSAGTPLYNRWAWD |
| | AGMSGLSGTTYNQGWRSWWLGSFNSSANNAQRVSGFSARS |
| | WLVDFATPPEPMPMTQVAAR |
| | MMKFDFDIDPLKIWATSGQFTQSSMDWHGATSTDLAAFRDR |
| | GGKMILYHGMSDAAFSALD |
| | TADYYERLGAAMPGAAGFARLFLVPGMNHCSGGPGTDRFD |
| | MLTPLVAWVERGEAPDQISA |
| | WSGTPGYFGVAARTRPLCPYPQIARYKGSGDINTEANFACAA |
| | PP |
| 49 | GGGSTPLPLPQQQPPQQEPPPPPVPLASRAACEALKDGNGDM |
| | VWPNAATVVEVAAWRDAA |
| | PATASAAALPEHCEVSGAIAKRTGIDGYPYEIKFRLRMPAEWN |
| | GRFFMEGGSGTNGSLSA |
| | ATGSIGGGQIASALSRGFAVIATDGGHDNAVNDNPDALGTVA |
| | FGLDPQARLDMGVNSYDQ |
| | VTQAGKAAVARFYGRAADKSYFIGCSNGGREGMMLSQRFPS |
| | HYDGIVAGAPGYQLPKAGI |
| | SGAWTTQSLAPAAVGLDAQGVPLINKSFSDADLHLLSQAILG |
| | TCDALDGLADGIVDNYRA |
| | CQAAFDPATAANPANGQALQCVGAKTADCLSPVQVTAIKRA |
| | MAGPVNSAGTPLYNRWAWD |
| | AGMSGLSGTTYNQGWRSWWLGSFNSSANNAQRVSGFSARS |
| | WLVDFATPPEPMPMTQVAAR |
| | MMKFDFDIDPLKIWATSGQFTQSSMDWHGATSTDLAAFRDR |
| | GGKMILYHGMSDAAFSALD |
| | TADYYERLGAAMPGAAGFARLFLVPGMNHCSGGPGTDRFD |
| | MLTPLVAWVERGEAPDQISA |
| | WSGTPGYFGVAARTRPLCPYPQIARYKGSGDINTEANFACAA |
| | PP |
| 50 | GGGSTPLPLPQQQPPQQEPPPPPVPLASRAACEALKDGNGDM |
| | VWPNAATVVEVAAWRDAA |
| | PATASAAALPEHCEVSGAIAKRTGIDGYPYEIKFRLRMPAEWN |
| | GRFFMEGGSGTNGSLSA |
| | ATGSIGGGQIASALSRGFAVIATDGGHDNAVNDNPDALGTVA |
| | FGLDPQARLDMGVNSYDQ |
| | VTQAGKAAVARFYGRAADKSYFIGCSEGGREGMMASQRFPS |
| | HYDGIVAGAPGYQLPKAGI |
| | SGAWTTQSLAPAAVGLDAQGVPLINKSFSDADLHLLSQAILG |
| | TCDALDGLADGIVDNYRA |
| | CQAAFDPATAANPANGQALQCVGAKTADCLSPVQVTAIKRA |
| | MAGPVNSAGTPLYNRWAWD |
| | AGMSGLSGTTYNQGWRSWWLGSFNSSANNAQRVSGFSARS |
| | WLVDFATPPEPMPMTQVAAR |
| | MMKFDFDIDPLKIWATSGQFTQSSMDWHGATSTDLAAFRDR |
| | GGKMILYHGMSDAAFSALD |
| | TADYYERLGAAMPGAAGFARLFLVPGMNHCSGGPGTDRFD |
| | MLTPLVAWVERGEAPDQISA |
| | WSGTPGYFGVAARTRPLCPYPQIARYKGSGDINTEANFACAA |
| | PP |
| 51 | GGGSTPLPLPQQQPPQQEPPPPPVPLASRAACEALKDGNGDM |
| | VWPNAATVVEVAAWRDAA |
| | PATASAAALPEHCEVSGAIAKRTGIDGYPYEIKFRLRMPAEWN |
| | GRFFMEGGSGTNGSLSA |
| | ATGSIGGGQIASALSRGFAVIATDGGHDNAVNDNPDALGTVA |
| | FGLDPQARLDMGVNSYDQ |
| | VTQAGKAAVARFYGRAADKSYFIGCSEGGREGMMLAQRFPS |
| | HYDGIVAGAPGYQLPKAGI |
| | SGAWTTQSLAPAAVGLDAQGVPLINKSFSDADLHLLSQAILG |
| | TCDALDGLADGIVDNYRA |
| | CQAAFDPATAANPANGQALQCVGAKTADCLSPVQVTAIKRA |
| | MAGPVNSAGTPLYNRWAWD |
| | AGMSGLSGTTYNQGWRSWWLGSFNSSANNAQRVSGFSARS |
| | WLVDFATPPEPMPMTQVAAR |
| | MMKFDFDIDPLKIWATSGQFTQSSMDWHGATSTDLAAFRDR |
| | GGKMILYHGMSDAAFSALD |
| | TADYYERLGAAMPGAAGFARLFLVPGMNHCSGGPGTDRFD |
| | MLTPLVAWVERGEAPDQISA |
| | WSGTPGYFGVAARTRPLCPYPQIARYKGSGDINTEANFACAA |
| | PP |
| 52 | GGGSTPLPLPQQQPPQQEPPPPPVPLASRAACEALKDGNGDM |
| | VWPNAATVVEVAAWRDAA |
| | PATASAAALPEHCEVSGAIAKRTGIDGYPYEIKFRLRMPAEWN |
| | GRFFMEGGSGTNGSLSA |
| | ATGSIGGGQIASALSRGFAVIATDGGHDNAVNDNPDALGTVA |
| | FGLDPQARLDMGVNSYDQ |
| | VTQAGKAAVARFYGRAADKSYFIGCSEGGREGMMLSQRFPS |
| | HYDGIVAGAPGYQLVKAGI |
| | SGAWTTQSLAPAAVGLDAQGVPLINKSFSDADLHLLSQAILG |
| | TCDALDGLADGIVDNYRA |
| | CQAAFDPATAANPANGQALQCVGAKTADCLSPVQVTAIKRA |
| | MAGPVNSAGTPLYNRWAWD |
| | AGMSGLSGTTYNQGWRSWWLGSFNSSANNAQRVSGFSARS |
| | WLVDFATPPEPMPMTQVAAR |
| | MMKFDFDIDPLKIWATSGQFTQSSMDWHGATSTDLAAFRDR |
| | GGKMILYHGMSDAAFSALD |
| | TADYYERLGAAMPGAAGFARLFLVPGMNHCSGGPGTDRFD |
| | MLTPLVAWVERGEAPDQISA |
| | WSGTPGYFGVAARTRPLCPYPQIARYKGSGDINTEANFACAA |
| | PP |
| 53 | GGGSTPLPLPQQQPPQQEPPPPPVPLASRAACEALKDGNGDM |
| | VWPNAATVVEVAAWRDAA |
| | PATASAAALPEHCEVSGAIAKRTGIDGYPYEIKFRLRMPAEWN |
| | GRFFMEGGSGTNGSLSA |
| | ATGSIGGGQIASALSRGFAVIATDGGHDNAVNDNPDALGTVA |
| | FGLDPQARLDMGVNSYDQ |
| | VTQAGKAAVARFYGRAADKSYFIGCSEGGREGMMLSQRFPS |
| | HYDGIVAGAPGYQLPKAAI |
| | SGAWTTQSLAPAAVGLDAQGVPLINKSFSDADLHLLSQAILG |
| | TCDALDGLADGIVDNYRA |
| | CQAAFDPATAANPANGQALQCVGAKTADCLSPVQVTAIKRA |
| | MAGPVNSAGTPLYNRWAWD |
| | AGMSGLSGTTYNQGWRSWWLGSFNSSANNAQRVSGFSARS |
| | WLVDFATPPEPMPMTQVAAR |
| | MMKFDFDIDPLKIWATSGQFTQSSMDWHGATSTDLAAFRDR |
| | GGKMILYHGMSDAAFSALD |
| | TADYYERLGAAMPGAAGFARLFLVPGMNHCSGGPGTDRFD |
| | MLTPLVAWVERGEAPDQISA |
| | WSGTPGYFGVAARTRPLCPYPQIARYKGSGDINTEANFACAA |
| | PP |
| 54 | GGGSTPLPLPQQQPPQQEPPPPPVPLASRAACEALKDGNGDM |
| | VWPNAATVVEVAAWRDAA |
| | PATASAAALPEHCEVSGAIAKRTGIDGYPYEIKFRLRMPAEWN |
| | GRFFMEGGSGTNGSLSA |
| | ATGSIGGGQIASALSRGFAVIATDGGHDNAVNDNPDALGTVA |
| | FGLDPQARLDMGVNSYDQ |
| | VTQAGKAAVARFYGRAADKSYFIGCSEGGREGMMLSQRFPS |
| | HYDGIVAGAPGYQLPKAGI |
| | AGAWTTQSLAPAAVGLDAQGVPLINKSFSDADLHLLSQAILG |
| | TCDALDGLADGIVDNYRA |
| | CQAAFDPATAANPANGQALQCVGAKTADCLSPVQVTAIKRA |
| | MAGPVNSAGTPLYNRWAWD |
| | AGMSGLSGTTYNQGWRSWWLGSFNSSANNAQRVSGFSARS |
| | WLVDFATPPEPMPMTQVAAR |
| | MMKFDFDIDPLKIWATSGQFTQSSMDWHGATSTDLAAFRDR |
| | GGKMILYHGMSDAAFSALD |
| | TADYYERLGAAMPGAAGFARLFLVPGMNHCSGGPGTDRFD |
| | MLTPLVAWVERGEAPDQISA |
| | WSGTPGYFGVAARTRPLCPYPQIARYKGSGDINTEANFACAA |
| | PP |
| 55 | GGGSTPLPLPQQQPPQQEPPPPPVPLASRAACEALKDGNGDM |
| | VWPNAATVVEVAAWRDAA |
| | PATASAAALPEHCEVSGAIAKRTGIDGYPYEIKFRLRMPAEWN |
| | GRFFMEGGSGTNGSLSA |
| | ATGSIGGGQIASALSRGFAVIATDGGHDNAVNDNPDALGTVA |
| | FGLDPQARLDMGVNSYDQ |
| | VTQAGKAAVARFYGRAADKSYFIGCSEGGREGMMLSQRFPS |
| | HYDGIVAGAPGYQLPKAGI |
| | SGAWLTQSLAPAAVGLDAQGVPLINKSFSDADLHLLSQAILG |
| | TCDALDGLADGIVDNYRA |
| | CQAAFDPATAANPANGQALQCVGAKTADCLSPVQVTAIKRA |
| | MAGPVNSAGTPLYNRWAWD |
| | AGMSGLSGTTYNQGWRSWWLGSFNSSANNAQRVSGFSARS |
| | WLVDFATPPEPMPMTQVAAR |
| | MMKFDFDIDPLKIWATSGQFTQSSMDWHGATSTDLAAFRDR |
| | GGKMILYHGMSDAAFSALD |
| | TADYYERLGAAMPGAAGFARLFLVPGMNHCSGGPGTDRFD |
| | MLTPLVAWVERGEAPDQISA |
| | WSGTPGYFGVAARTRPLCPYPQIARYKGSGDINTEANFACAA |
| | PP |
| 56 | GGGSTPLPLPQQQPPQQEPPPPPVPLASRAACEALKDGNGDM |
| | VWPNAATVVEVAAWRDAA |
| | PATASAAALPEHCEVSGAIAKRTGIDGYPYEIKFRLRMPAEWN |
| | GRFFMEGGSGTNGSLSA |
| | ATGSIGGGQIASALSRGFAVIATDGGHDNAVNDNPDALGTVA |
| | FGLDPQARLDMGVNSYDQ |
| | VTQAGKAAVARFYGRAADKSYFIGCSEGGREGMMLSQRFPS |
| | HYDGIVAGAPGYQLPKAGI |
| | SGAWTLQSLAPAAVGLDAQGVPLINKSFSDADLHLLSQAILG |
| | TCDALDGLADGIVDNYRA |
| | CQAAFDPATAANPANGQALQCVGAKTADCLSPVQVTAIKRA |
| | MAGPVNSAGTPLYNRWAWD |
| | AGMSGLSGTTYNQGWRSWWLGSFNSSANNAQRVSGFSARS |
| | WLVDFATPPEPMPMTQVAAR |
| | MMKFDFDIDPLKIWATSGQFTQSSMDWHGATSTDLAAFRDR |
| | GGKMILYHGMSDAAFSALD |
| | TADYYERLGAAMPGAAGFARLFLVPGMNHCSGGPGTDRFD |
| | MLTPLVAWVERGEAPDQISA |
| | WSGTPGYFGVAARTRPLCPYPQIARYKGSGDINTEANFACAA |
| | PP |
| 57 | GGGSTPLPLPQQQPPQQEPPPPPVPLASRAACEALKDGNGDM |
| | VWPNAATVVEVAAWRDAA |
| | PATASAAALPEHCEVSGAIAKRTGIDGYPYEIKFRLRMPAEWN |
| | GRFFMEGGSGTNGSLSA |
| | ATGSIGGGQIASALSRGFAVIATDGGHDNAVNDNPDALGTVA |
| | FGLDPQARLDMGVNSYDQ |
| | VTQAGKAAVARFYGRAADKSYFIGCSEGGREGMMLSQRFPS |
| | HYDGIVAGAPGYQLPKAGI |
| | SGAWTTQSLAPAAVGLDAQGVPLILKSFSDADLHLLSQAILGT |
| | CDALDGLADGIVDNYRA |
| | CQAAFDPATAANPANGQALQCVGAKTADCLSPVQVTAIKRA |
| | MAGPVNSAGTPLYNRWAWD |
| | AGMSGLSGTTYNQGWRSWWLGSFNSSANNAQRVSGFSARS |
| | WLVDFATPPEPMPMTQVAAR |
| | MMKFDFDIDPLKIWATSGQFTQSSMDWHGATSTDLAAFRDR |
| | GGKMILYHGMSDAAFSALD |
| | TADYYERLGAAMPGAAGFARLFLVPGMNHCSGGPGTDRFD |
| | MLTPLVAWVERGEAPDQISA |
| | WSGTPGYFGVAARTRPLCPYPQIARYKGSGDINTEANFACAA |
| | PP |
| 58 | GGGSTPLPLPQQQPPQQEPPPPPVPLASRAACEALKDGNGDM |
| | VWPNAATVVEVAAWRDAA |
| | PATASAAALPEHCEVSGAIAKRTGIDGYPYEIKFRLRMPAEWN |
| | GRFFMEGGSGTNGSLSA |
| | ATGSIGGGQIASALSRGFAVIATDGGHDNAVNDNPDALGTVA |
| | FGLDPQARLDMGVNSYDQ |
| | VTQAGKAAVARFYGRAADKSYFIGCSEGGREGMMLSQRFPS |
| | HYDGIVAGAPGYQLPKAGI |
| | SGAWTTQSLAPAAVGLDAQGVPLINKSFSDADLHLVSQAILG |
| | TCDALDGLADGIVDNYRA |
| | CQAAFDPATAANPANGQALQCVGAKTADCLSPVQVTAIKRA |
| | MAGPVNSAGTPLYNRWAWD |
| | AGMSGLSGTTYNQGWRSWWLGSFNSSANNAQRVSGFSARS |
| | WLVDFATPPEPMPMTQVAAR |
| | MMKFDFDIDPLKIWATSGQFTQSSMDWHGATSTDLAAFRDR |
| | GGKMILYHGMSDAAFSALD |
| | TADYYERLGAAMPGAAGFARLFLVPGMNHCSGGPGTDRFD |
| | MLTPLVAWVERGEAPDQISA |
| | WSGTPGYFGVAARTRPLCPYPQIARYKGSGDINTEANFACAA |
| | PP |
| 59 | GGGSTPLPLPQQQPPQQEPPPPPVPLASRAACEALKDGNGDM |
| | VWPNAATVVEVAAWRDAA |
| | PATASAAALPEHCEVSGAIAKRTGIDGYPYEIKFRLRMPAEWN |
| | GRFFMEGGSGTNGSLSA |
| | ATGSIGGGQIASALSRGFAVIATDGGHDNAVNDNPDALGTVA |
| | FGLDPQARLDMGVNSYDQ |
| | VTQAGKAAVARFYGRAADKSYFIGCSEGGREGMMLSQRFPS |
| | HYDGIVAGAPGYQLPKAGI |
| | SGAWTTQSLAPAAVGLDAQGVPLINKSFSDADLHLLAQAILG |
| | TCDALDGLADGIVDNYRA |
| | CQAAFDPATAANPANGQALQCVGAKTADCLSPVQVTAIKRA |
| | MAGPVNSAGTPLYNRWAWD |
| | AGMSGLSGTTYNQGWRSWWLGSFNSSANNAQRVSGFSARS |
| | WLVDFATPPEPMPMTQVAAR |
| | MMKFDFDIDPLKIWATSGQFTQSSMDWHGATSTDLAAFRDR |
| | GGKMILYHGMSDAAFSALD |
| | TADYYERLGAAMPGAAGFARLFLVPGMNHCSGGPGTDRFD |
| | MLTPLVAWVERGEAPDQISA |
| | WSGTPGYFGVAARTRPLCPYPQIARYKGSGDINTEANFACAA |
| | PP |
| 60 | GGGSTPLPLPQQQPPQQEPPPPPVPLASRAACEALKDGNGDM |
| | VWPNAATVVEVAAWRDAA |
| | PATASAAALPEHCEVSGAIAKRTGIDGYPYEIKFRLRMPAEWN |
| | GRFFMEGGSGTNGSLSA |
| | ATGSIGGGQIASALSRGFAVIATDGGHDNAVNDNPDALGTVA |
| | FGLDPQARLDMGVNSYDQ |
| | VTQAGKAAVARFYGRAADKSYFIGCSEGGREGMMLSQRFPS |
| | HYDGIVAGAPGYQLPKAGI |
| | SGAWTTQSLAPAAVGLDAQGVPLINKSFSDADLHLLSQAILG |
| | TCDALDGLADGIVDNYRA |
| | CQAAFDPATAANPANGQALQCVGAKTADCLSPVQVRAIKRA |
| | MAGPVNSAGTPLYNRWAWD |
| | AGMSGLSGTTYNQGWRSWWLGSFNSSANNAQRVSGFSARS |
| | WLVDFATPPEPMPMTQVAAR |
| | MMKFDFDIDPLKIWATSGQFTQSSMDWHGATSTDLAAFRDR |
| | GGKMILYHGMSDAAFSALD |
| | TADYYERLGAAMPGAAGFARLFLVPGMNHCSGGPGTDRFD |
| | MLTPLVAWVERGEAPDQISA |
| | WSGTPGYFGVAARTRPLCPYPQIARYKGSGDINTEANFACAA |
| | PP |
| 61 | GGGSTPLPLPQQQPPQQEPPPPPVPLASRAACEALKDGNGDM |
| | VWPNAATVVEVAAWRDAA |
| | PATASAAALPEHCEVSGAIAKRTGIDGYPYEIKFRLRMPAEWN |
| | GRFFMEGGSGTNGSLSA |
| | ATGSIGGGQIASALSRGFAVIATDGGHDNAVNDNPDALGTVA |
| | FGLDPQARLDMGVNSYDQ |
| | VTQAGKAAVARFYGRAADKSYFIGCSEGGREGMMLSQRFPS |
| | HYDGIVAGAPGYQLPKAGI |
| | SGAWTTQSLAPAAVGLDAQGVPLINKSFSDADLHLLSQAILG |
| | TCDALDGLADGIVDNYRA |
| | CQAAFDPATAANPANGQALQCVGAKTADCLSPVQVTAIKRA |
| | MAGPVNSAGTPLYNRWPWD |
| | AGMSGLSGTTYNQGWRSWWLGSFNSSANNAQRVSGFSARS |
| | WLVDFATPPEPMPMTQVAAR |
| | MMKFDFDIDPLKIWATSGQFTQSSMDWHGATSTDLAAFRDR |
| | GGKMILYHGMSDAAFSALD |
| | TADYYERLGAAMPGAAGFARLFLVPGMNHCSGGPGTDRFD |
| | MLTPLVAWVERGEAPDQISA |
| | WSGTPGYFGVAARTRPLCPYPQIARYKGSGDINTEANFACAA |
| | PP |
| 62 | GGGSTPLPLPQQQPPQQEPPPPPVPLASRAACEALKDGNGDM |
| | VWPNAATVVEVAAWRDAA |
| | PATASAAALPEHCEVSGAIAKRTGIDGYPYEIKFRLRMPAEWN |
| | GRFFMEGGSGTNGSLSA |
| | ATGSIGGGQIASALSRGFAVIATDGGHDNAVNDNPDALGTVA |
| | FGLDPQARLDMGVNSYDQ |
| | VTQAGKAAVARFYGRAADKSYFIGCSEGGREGMMLSQRFPS |
| | HYDGIVAGAPGYQLPKAGI |
| | SGAWTTQSLAPAAVGLDAQGVPLINKSFSDADLHLLSQAILG |
| | TCDALDGLADGIVDNYRA |
| | CQAAFDPATAANPANGQALQCVGAKTADCLSPVQVTAIKRA |
| | MAGPVNSAGTPLYNRWAWD |
| | AGMSGLSGTTYNQGWRSWWLGSFNSSANNAQRVSGFSARS |
| | WLVDFATPPEPMPMTQVAAR |
| | MMKFDFDIDPLKIWATSGQFTQSLMDWHGATSTDLAAFRDR |
| | GGKMILYHGMSDAAFSALD |
| | TADYYERLGAAMPGAAGFARLFLVPGMNHCSGGPGTDRFD |
| | MLTPLVAWVERGEAPDQISA |
| | WSGTPGYFGVAARTRPLCPYPQIARYKGSGDINTEANFACAA |
| | PP |
| 63 | GGGSTPLPLPQQQPPQQEPPPPPVPLASRAACEALKDGNGDM |
| | VWPNAATVVEVAAWRDAA |
| | PATASAAALPEHCEVSGAIAKRTGIDGYPYEIKFRLRMPAEWN |
| | GRFFMEGGSGTNGSLSA |
| | ATGSIGGGQIASALSRGFAVIATDGGHDNAVNDNPDALGTVA |
| | FGLDPQARLDMGVNSYDQ |
| | VTQAGKAAVARFYGRAADKSYFIGCSEGGREGMMLSQRFPS |
| | HYDGIVAGAPGYQLPKAGI |
| | SGAWTTQSLAPAAVGLDAQGVPLINKSFSDADLHLLSQAILG |
| | TCDALDGLADGIVDNYRA |
| | CQAAFDPATAANPANGQALQCVGAKTADCLSPVQVTAIKRA |
| | MAGPVNSAGTPLYNRWAWD |
| | AGMSGLSGTTYNQGWRSWWLGSFNSSANNAQRVSGFSARS |
| | WLVDFATPPEPMPMTQVAAR |
| | MMKFDFDIDPLKIWATSGQFTQSSMDWHGATSTDLAAFRDR |
| | GGKMILYHGMSDPAFSALD |
| | TADYYERLGAAMPGAAGFARLFLVPGMNHCSGGPGTDRFD |
| | MLTPLVAWVERGEAPDQISA |
| | WSGTPGYFGVAARTRPLCPYPQIARYKGSGDINTEANFACAA |
| | PP |
| 64 | GGGSTPLPLPQQQPPQQEPPPPPVPLASRAACEALKDGNGDM |
| | VWPNAATVVEVAAWRDAA |
| | PATASAAALPEHCEVSGAIAKRTGIDGYPYEIKFRLRMPAEWN |
| | GRFFMEGGSGTNGSLSA |
| | ATGSIGGGQIASALSRGFAVIATDGGHDNAVNDNPDALGTVA |
| | FGLDPQARLDMGVNSYDQ |
| | VTQAGKAAVARFYGRAADKSYFIGCSEGGREGMMLSQRFPS |
| | HYDGIVAGAPGYQLPKAGI |
| | SGAWTTQSLAPAAVGLDAQGVPLINKSFSDADLHLLSQAILG |
| | TCDALDGLADGIVDNYRA |
| | CQAAFDPATAANPANGQALQCVGAKTADCLSPVQVTAIKRA |
| | MAGPVNSAGTPLYNRWAWD |
| | AGMSGLSGTTYNQGWRSWWLGSFNSSANNAQRVSGFSARS |
| | WLVDFATPPEPMPMTQVAAR |
| | MMKFDFDIDPLKIWATSGQFTQSSMDWHGATSTDLAAFRDR |
| | GGKMILYHGMSDAAFSALD |
| | TADWYERLGAAMPGAAGFARLFLVPGMNHCSGGPGTDRFD |
| | MLTPLVAWVERGEAPDQISA |
| | WSGTPGYFGVAARTRPLCPYPQIARYKGSGDINTEANFACAA |
| | PP |
| 65 | GGGSTPLPLPQQQPPQQEPPPPPVPLASRAACEALKDGNGDM |
| | VWPNAATVVEVAAWRDAA |
| | PATASAAALPEHCEVSGAIAKRTGIDGYPYEIKFRLRMPAEWN |
| | GRFFMEGGSGINGSLSA |
| | ATGSIGGGQIASALSRGFAVIATDGGHDNAVNDNPDALGTVA |
| | FGLDPQARLDMGVNSYDQ |
| | VTQAGKAAVARFYGRAADKSYFIGCSEGGREGMMLSQRFPS |
| | HYDGIVAGAPGYQLPKAGI |
| | SGAWTTQSLAPAAVGLDAQGVPLINKSFSDADLHLLSQAILG |
| | TCDALDGLADGIVDNYRA |
| | CQAAFDPATAANPANGQALQCVGAKTADCLSPVQVTAIKRA |
| | MAGPVNSAGTPLYNRWAWD |
| | AGMSGLSGTTYNQGWRSWWLGSFNSSANNAQRVSGFSARS |
| | WLVDFATPPEPMPMTQVAAR |
| | MMKFDFDIDPLKIWATSGQFTQSSMDWHGATSTDLAAFRDR |
| | GGKMILYHGMSDAAFSALD |
| | TADYYERLGAAMPGAAGFARLFLVPGMNHCSGGPGTDRFD |
| | MLTPLVAWVERGEAPDQISA |
| | WSGTPGYFGVAARTRPLCPYPQIARYKGSGDINTAANFACAA |
| | PP |
| 66 | GGGSTPLPLPQQQPPQQEPPPPPVPLASRAACEALKDGNGDM |
| | VWPNAATVVEVAAWRDAA |
| | PATASAAALPEHCEVSGAIAKRTGIDGYPYEIKFRLRMPAEWN |
| | GRFFMEGGSGTNGSLSA |
| | ATGSIGGGQIASALSRGFAVIATDGGHDNAVNDNPDALGTVA |
| | FGLDPQARLDMGVNSYDQ |
| | VTQAGKAAVARFYGRAADKSYFIGCSEGGREGMMLSQRFPS |
| | HYDGIVAGAPGYQLPKAGI |
| | SGAWTTQSLAPAAVGLDAQGVPLINKSFSDADLHLLSQAILG |
| | TCDALDGLADGIVDNYRA |
| | CQAAFDPATAANPANGQALQCVGAKTADCLSPVQVTAIKRA |
| | MAGPVNSAGTPLYNRWAWD |
| | AGMSGLSGTTYNQGWRSWWLGSFNSSANNAQRVSGFSARS |
| | WLVDFATPPEPMPMTQVAAR |
| | MMKFDFDIDPLKIWATSGQFTQSSMDWHGATSTDLAAFRDR |
| | GGKMILYHGMSDAAFSALD |
| | TADYYERLGAAMPGAAGFARLFLVPGMNHCSGGPGTDRFD |
| | MLTPLVAWVERGEAPDQISA |
| | WSGTPGYFGVAARTRPLCPYPQIARYKGSGDINTEASFACAAP |
| | P |
| 67 | GGGSTPLPLPQQQPPQQEPPPPPVPLASRAA |
| | CEALKDGNGDMVWPNAATVVEVAAWRDAAPATASAAALPE |
| | HCEVSGAIAKRTGIDGYPYE |
| | IKFRLRMPAEWNGRFFMEGGSGTNGSLSAATGSIGGGQIASAL |
| | SRGFAVIATDGGHDNAV |
| | NDNPDALGTVAFGLDPQARLDMGYNSYDQVTQAGKAAVAR |
| | FYGRAADKSYFIGCSEGGRE |
| | GMMLSQRFPSHYDGIVAGAPGYQLPKAGISGAWTTQSLAPAA |
| | VGLDAQGVPLINKSFSDA |
| | DLHLLSQAILGTCDALDGLADGIVDNYRACQAAFDPATAANP |
| | ANGQALQCVGAKTADCLS |
| | PVQVTAIKRAMAGPVNSAGTPLYNRWAWDAGMSGLSGTTY |
| | NQGWRSWWLGSFNSSANNAQ |
| | RVSGFSARSWLVDFATPPEPMPMTQVAARMMKFDFDIDPLKI |
| | WATSGQFTQSSMDWHGAT |
| | STDLAAFRDRGGKMILYHGMSDAAFSALDTADYYERLGAAM |
| | PGAAGFARLFLVPGMNHCS |
| | GGPGTDRFDMLTPLVAWVERGEAPDQISAWSGTPGYFGVAA |
| | RTRPLCPYPQIARYKGSGD |
| | INTEANFACAAPP |
| 68 | CEALKDGNGDMVWPNAATVVEVAAWRDAAPATASAAALPE |
| | HCEVSGAIAKRTGIDGYPYE |
| | IKFRLRMPAEWNGRFFMEGGSGTNGSLSAATGSIGGGQIASAL |
| | SRGFAVIATDGGHDNAV |
| | NDNPDALGTVAFGLDPQARLDMGYNSYDQVTQAGKAAVAR |
| | FYGRAADKSYFIGCSEGGRE |
| | GMMLSQRFPSHYDGIVAGAPGYQLPKAGISGAWTTQSLAPAA |
| | VGLDAQGVPLINKSFSDA |
| | DLHLLSQAILGTCDALDGLADGIVDNYRACQAAFDPATAANP |
| | ANGQALQCVGAKTADCLS |
| | PVQVTAIKRAMAGPVNSAGTPLYNRWAWDAGMSGLSGTTY |
| | NQGWRSWWLGSFNSSANNAQ |
| | RVSGFSARSWLVDFATPPEPMPMTQVAARMMKFDFDIDPLKI |
| | WATSGQFTQSSMDWHGAT |
| | STDLAAFRDRGGKMILYHGMSDAAFSALDTADWYERLGAA |
| | MPGAAGFARLFLVPGMNHCS |
| | GGPGTDRFDMLTPLVAWVERGEAPDQISAWSGTPGYFGVAA |
| | RTRPLCPYPQIARYKGSGD |
| | INTEANFACAAPP |
| 69 | GGGSTPLPLPQQQPPQQEPPPPPVPLASRAA |
| | CEALKDGNGDMVWPNAATVVEVAAWRDAAPATASAAALPE |
| | HCEVSGAIAKRTGIDGYPYE |
| | IKFRLRMPAEWNGRFFMEGGSGTNGSLSAATGSIGGGQIASAL |
| | SRGFAVIATDGGHDNAV |
| | NDNPDALGTVAFGLDPQARLDYGYNSYDQVTQAGKAAVAR |
| | FYGRAADKSYFIGCSEGGRE |
| | GMMLSQRFPSHYDGIVAGAPGYQLPKAGISGAWTTQSLAPAA |
| | VGLDAQGVPLINKSFSDA |
| | DLHLLSQAILGTCDALDGLADGIVDNYRACQAAFDPATAANP |
| | ANGQALQCVGAKTADCLS |
| | PVQVTAIKRAMAGPVNSAGTPLYNRWAWDAGMSGLSGTTY |
| | NQGWRSWWLGSFNSSANNAQ |
| | RVSGFSARSWLVDFATPPEPMPMTQVAARMMKFDFDIDPLKI |
| | WATSGQFTQSSMDWHGAT |
| | STDLAAFRDRGGKMILYHGMSDAAFSALDTADWYERLGAA |
| | MPGAAGFARLFLVPGMNHCS |
| | GGPGTDRFDMLTPLVAWVERGEAPDQISAWSGTPGYFGVAA |
| | RTRPLCPYPQIARYKGSGD |
| | INTEANFACAAPP |
| 70 | GGGSTPLPLPQQQPPQQEPPPPPVPLASRAA |
| | CEALKDGNGDMVWPNAATVVEVAAWRDAAPATASAAALPE |
| | HCEVSGAIAKRTGIDGYPYE |
| | IKFRLRMPAEWNGRFFMEGGSGTNGSLSAATGSIGGGQIASAL |
| | SRNFAVIATDGGHDNAV |
| | NDNPDALGTVAFGLDPQARLDYGYNSYDQVTQAGKAAVAR |
| | FYGRAADKSYFIGCSEGGRE |
| | GMMLSQRFPSHYDGIVAGAPGYQLPKAGISGAWTTQSLAPAA |
| | VGLDAQGVPLINKSFSDA |
| | DLHLLSQAILGTCDALDGLADGIVDNYRACQAAFDPATAANP |
| | ANGQALQCVGAKTADCLS |
| | PVQVTAIKRAMAGPVNSAGTPLYNRWAWDAGMSGLSGTTY |
| | NQGWRSWWLGSFNSSANNAQ |
| | RVSGFSARSWLVDFATPPEPMPMTQVAARMMKFDFDIDPLKI |
| | WATSGQFTQSSMDWHGAT |
| | STDLAAFRDRGGKMILYHGMSDAAFSALDTADWYERLGAA |
| | MPGAAGFARLFLVPGMNHCS |
| | GGPGTDRFDMLTPLVAWVERGEAPDQISAWSGTPGYFGVAA |
| | RTRPLCPYPQIARYKGSGD |
| | INTEANFACAAPP |
| 71 | GGGSTPLPLPQQQPPQQEPPPPPVPLASRAA |
| | CEALKDGNGDMVWPNAATVVEVAAWRDAAPATASAAALPE |
| | HCEVSGAIAKRTGIDGYPYE |
| | IKFRLRMPAEWNGRFFMEGGSGTNGSLSAATGSIGGGQIASAL |
| | SRNFAVIATDGGHDNAV |
| | NDNPDALGTVAFGLDPQARLDYGYNSYDQVTQAGKAAVAR |
| | FYGRAADKSYFIGCSEGGRE |
| | GMMLSQRFPSHYDGIVAGAPGFQLPKAGISGAWTTQSLAPAA |
| | VGLDAQGVPLINKSFSDA |
| | DLHLLSQAILGTCDALDGLADGIVDNYRACQAAFDPATAANP |
| | ANGQALQCVGAKTADCLS |
| | PVQVTAIKRAMAGPVNSAGTPLYNRWAWDAGMSGLSGTTY |
| | NQGWRSWWLGSFNSSANNAQ |
| | RVSGFSARSWLVDFATPPEPMPMTQVAARMMKFDFDIDPLKI |
| | WATSGQFTQSSMDWHGAT |
| | STDLAAFRDRGGKMILYHGMSDAAFSALDTADWYERLGAA |
| | MPGAAGFARLFLVPGMNHCS |
| | GGPGTDRFDMLTPLVAWVERGEAPDQISAWSGTPGYFGVAA |
| | RTRPLCPYPQIARYKGSGD |
| | INTEANFACAAPP |
| 72 | GGGSTPLPLPQQQPPQQEPPPPPVPLASRAA |
| | CEALKDGNGDMVWPNAATVVEVAAWRDAAPATASAAALPE |
| | HCEVSGAIAKRTGIDGYPYE |
| | IKFRLRMPAEWNGRFFMEGGSGTNGSLSAATGSIGGGQIASAL |
| | SRNFAVIATDGGHDNAV |
| | NDNPDALGTVAFGLDPQARLDMGYNSYDQVTQAGKAAVAR |
| | FYGRAADKSYFIGCSEGGRE |
| | GMMLSQRFPSHYDGIVAGAPGFQLPKAGISGAWTTQSLAPAA |
| | VGLDAQGVPLINKSFSDA |
| | DLHLLSQAILGTCDALDGLADGIVDNYRACQAAFDPATAANP |
| | ANGQALQCVGAKTADCLS |
| | PVQVTAIKRAMAGPVNSAGTPLYNRWAWDAGMSGLSGTTY |
| | NQGWRSWWLGSFNSSANNAQ |
| | RVSGFSARSWLVDFATPPEPMPMTQVAARMMKFDFDIDPLKI |
| | WATSGQFTQSSMDWHGAT |
| | STDLAAFRDRGGKMILYHGMSDAAFSALDTADWYERLGAA |
| | MPGAAGFARLFLVPGMNHCS |
| | GGPGTDRFDMLTPLVAWVERGEAPDQISAWSGTPGYFGVAA |
| | RTRPLCPYPQIARYKGSGD |
| | INTEANFACAAPP |
| 73 | MLLPETRNLLDLMDAATRGGRPRLETLPHAVGRKAVDKMSE |
| | DGEADPPEVAEVANGGFAGPASEIRFRRYRPLGEAAGLLPTLI |
| | YYHGGGFVIGNIETHDSTCRRLANKSRCQVISIDYRLAPEHPFP |
| | APIDDGIAAFRHIRDNAESFGADAARLAVGGDSAGGAMAAV |
| | VCQACRDAGETGPAFQMLIYPATDSSRESASRVAFAEGYFLS |
| | KALMDWFWEAYVPEDTDLTDLRLSPLLATDFTGLPPAFVLTA |
| | GYDPLRDEGRAYADRLIEAGIKTTYVNYPGTIHGFFSLTRFLS |
| | QGLKANDEAAAVMGAHFGT* |
| 74 | MLDHEARVLLELMEKAVQEGQPKLHTLPYAVGRQAVDKMS |
| | EDSEADPMQVGEVADGAFAGPGGEIRFRRYRPLGAAAGPLPT |
| | LIYYHGGGFVIGNIETHDSTCRRLANKSRCQVISIDYRLSPEHP |
| | FPASTDDGIAAFRHVRDNAASFGVDATRIAVGGDSAGGAIAA |
| | VVCQAIRDAGDKQGPAFQMLIYPATDSSKQSASRVAFAEGYF |
| | LTKELMEWFWKAYVPAGTDLSDLRLSPLLAEDFRGLPPAFVL |
| | TAGYDPLRDEGRAYADRLIDAGIKTTYVNYPGTIHGFFSLTRF |
| | LSQGLKANDEAAAVMAAHFG* |
| 75 | MLDQEARTLLDLMEEANKAGRPKLETLPHALGRAAVDKMSE |
| | DGEAEPMDVAEVADGGFMGPGGEIRFRKYRPLGVNAGSLPT |
| | LIYYHGGGFVIGTIETHDSTCRRLANKSRCQVISIAYRLAPEHP |
| | FPAPTDDAVAAFRHIRDNAASFGADPGRLAVGGDSAGGALA |
| | AVVCQAMRDAQEAGPAFQMLIYPATDSSKESGSRKAFAEGY |
| | FLTKSLMDWFWKAYVPAGTDLADLRLSPLLAKSFEGLPPAFV |
| | LTAGFDPLRDEGRAYADRLIDAGVKTTYVNYPGTIHGFFSMT |
| | RFLAQGLRANDEAAAVLGAHFG* |
| 76 | MLDAEARILLDLMEEATKAGRPKLETLPHAVGRAAVDKMSE |
| | DSEADPPQVGDTVDGVFAGPGGPIKYRRYLPLGVTLALTQGN |
| | LPTLIYYHGGGFVIGTIETHDSTCRRLANKSRCQVISIDYRLSP |
| | EHPFPAPIDDGIAAFRHIRDHADTFGADPARLAVGGDSAGGAV |
| | AAVVCQAMRDAKEAGPAFQMLIYPATDSSKESGSRQQFAEG |
| | YFLTKGLMDWFWKAYVPAGTDLSDLRLSPLLAKDFTGLPPAF |
| | VLTAGHDPLRDEGRAYANRLIDAGVKTTYVNYPGTIHGFFSL |
| | TRFLQQGLKANDEAAAVMGAFFG* |
| 77 | MLDAEARILLDLMDKAVKEGRPLLHTLPHKVGRAAVDKMSE |
| | DSEADPPAVGEVTDGAFAGPDGEIRFRHYRPFGVPSGPLPTLV |
| | YYHGGGFVIGNLETHDSTCRRLANKSRCQVIAVDYRLSPEYPF |
| | PAPIDDGVAAFRHIRDHAASFGADAKRIAVGGDSAGGAMAA |
| | VVCQTCRDAGDAPPAFQMLIYPATDSSKESRSRRELAEGYFLT |
| | KDLMDWFWSAYVPAGTDLSDIRLSPLLARDFKGLPPAFVLTA |
| | GHDPLRDEGRAYADRLIDAGVKTTYVNYPGTIHGFFSLTRFL |
| | QQGLKANDEAAAVLAAHFG* |

As used herein, the term “sequence identity” or “identity” refers to the number (or fraction expressed as a percentage %) of matches (identical amino acid residues) between two polypeptide sequences. In a preferred embodiment, the sequence identity is determined by comparing the sequences when aligned so as to maximize overlap and identity while minimizing sequence gaps. Sequence identity may be determined using any of a number of mathematical global or local alignment algorithms known to persons skilled in the art, depending on the length of the two sequences. Sequences of similar lengths may be aligned using a global alignment algorithms (e.g., Needleman and Wunsch algorithm; Needleman and Wunsch, 1970), which aligns the sequences optimally over the entire length, while sequences of substantially different lengths are preferably aligned using a local alignment algorithm (e.g., Smith and Waterman algorithm (Smith and Waterman, 1981) or Altschul algorithm (Altschul et al., 1997; Altschul et al., 2005)). Alignment for the purposes of determining percent amino acid sequence identity can be achieved by any means available to persons skilled in the art, illustrative examples of which include publicly available computer software, such as is available at http://blast.ncbi.nlm.nih.gov/ or http://www.ebi.ae.uk/Tools/emboss/). Persons skilled in the art can readily determine appropriate parameters for measuring alignment, including any algorithms needed to achieve maximal alignment over the full length of the sequences being compared. As used herein, % sequence identity typically refers to values generated using pair wise sequence alignment that creates an optimal global alignment of two sequences (e.g., using the Needleman-Wunsch algorithm), where all search parameters are set to default values, e.g., Scoring matrix=BLOSUM62, Gap open=10, Gap extend=0.5, End gap penalty=false, End gap open=10 and End gap extend=0.5.

As noted elsewhere herein, the esterase, including variants of extant esterases, will suitably retain at least some esterase activity, irrespective of any modifications made, including to its amino acid sequence. Suitable methods of determining or measuring esterase/hydrolase activity will be familiar to persons skilled in the art, illustrative examples of which are described in Palm et al. (2019, Nat. Comm., 10:1717), Sagong et al. (2020, ACS Catal. 10:4805) and Yoshida et al. (2016, Science, 352 (6278): 1196), the contents of which are incorporated herein by reference in their entirety. In an embodiment, the esterase/hydrolase activity is determined by UV absorbance assay to monitor the amount of product or terephthalic acid produced from using amorphous PET as a substrate. Another method useful for determining or measuring the esterase/hydrolase activity is by measuring the amount of terephthalic acid produced using Analytical High Performance Liquid Chromatography (HPLC). The hydrolase activity of the esterase may be assigned an absolute value or a value relative to the hydrolase activity of a comparator (e.g., an extant PETase or cutinases having PETase activity). In an embodiment, the esterase/hydrolase activity is measured as the rate of monomers and/or oligomers (e.g., in mg or mol) released per hour and per mg or mol of enzyme under suitable conditions of temperature, pH and buffer. In an embodiment, the rate of monomers and/or oligomers released per hour is in the range of from about 1 mol/h/mol enzyme to about 500 mol/h/mol enzyme. In another embodiment, the rate of monomers and/or oligomers (e.g., in mg) released per hour is in the range of from about 50 mol/h/mol enzyme to about 400 mol/h/mol enzyme. In yet another embodiment, the rate of monomers and/or oligomers (e.g., in mg) released per hour is in the range of from about 100 mol/h/mol enzyme to about 350 mol/h/mol enzyme.

In an embodiment, the esterase is capable of catalysing the hydrolysis of mono- and di-, and poly-terephthalic acid esters at least in a range of temperatures from about 0° C. to about 80° C., for example from about 2° C. to 75° C., from about 4° C. to 70° C., from about 6° C. to 70° C.: from about 8° C. to 65° C., from about 10° C. to 65° C., from about 20° C. to 60° C., from about 25° C. to 55° C. from about 30° C. to 55° C., from about 30° C. to 50° C., or about 60° C., or about 50° C. In an embodiment, the esterase described herein exhibits hydrolase activity at a temperature from about 0° C. to about 80° C., for example from about 2° C. to 80° C., from about 4° C. to 80° C., from about 6° C. to 80° C.: from about 8° C. to 80° C., from about 10° C. to about 80° C., or from about 20° C. to about 80° C., or from about 30° C. to about 80° C., or from about 40° C. to about 80° C., or from about 50° C. to about 80° C., or from about 60° C. to about 80° C., or from about 60° C. to about 70° C., or at about 60° C. or at about 50° C. In an embodiment, the hydrolase activity of the esterase is measurable at a temperature from about 0° C. to about 80° C., for example from about 2° C. to 80° C., from about 4° C. to 80° C., from about 6° C. to 80° C.: from about 8° C. to 80° C., 10° C. to about 70° C., or from about 20° C. to about 80° C., or from about 30° C. to about 80° C., or from about 40° C. to about 80° C., or from about 50° C. to about 80° C., or from about 60° C. to about 80° C., or from about 60° C. to about 70° C., or at about 60° C. In another particular embodiment, the hydrolase/esterase activity is still measurable at a temperature from about 10° C. to about 30° C., or about 15° C. to about 28° C., corresponding to the mean temperature in the natural environment (ambient temperature).

In an embodiment, the esterase exhibits measurable hydrolase/esterase activity at least in a range of pH from about 5 to about 11, or in a range of pH from about 6 to about 10, or in a range of pH from about 7 to about 10, or in a range of pH from about 7.5 to about 9.5.

The amount of esterase used will of course depend upon the amount of the one or both of a mono-ester and diester terephthalate C₆-C₁₀ mono-alcohol derivative to be converted into terephthalic acid. Generally, the amount of esterase used will range from 1:1000 to about 1:10 (enzyme mass to crude transesterification product mass (step (i))).

If required, one or more additives can be combined with the esterase and transesterification product produced in step (i) to assist with stabilising the esterase and/or increase the rate of esterase activity. Such additives are known to those skilled in the art and might include, but are not limited to, calcium chloride and ammonium sulfate.

Once the enzymatic conversion into terephthalic acid is complete, it may be desirable to subject the composition produced in step (ii) to one or more purification procedures to increase the purity of the so formed terephthalic acid.

The purity of the terephthalic acid produced in step (ii) may be increased using techniques well-known to those skilled in the art. For example, the composition comprising terephthalic acid produced in step (ii) may be subjected to one or more techniques selected from washing, solvent extraction, filtration, distillation, solvent evaporation, column chromatography and crystallisation.

In one embodiment, step (ii) produces a composition comprising terephthalic acid that is subjected to one or more techniques to increase the purity of the so formed terephthalic acid.

In another embodiment, the one or more techniques used to increase the purity of the so formed terephthalic acid are selected from washing, solvent extraction, filtration, distillation, solvent evaporation, column chromatography and crystallisation.

In a further embodiment, the terephthalic acid produced in step (ii) is purified and isolated.

Production of the terephthalic acid can be confirmed using techniques well-known to those skilled in the art. For example, the presence of terephthalic acid can be readily determined using NMR spectroscopy.

It may be convenient to perform the method of the invention using esterase that is immobilised on a substrate. Using immobilised esterase may be beneficial when performing the method of the invention in a semicontinuous or continuous manner.

In one embodiment, the esterase is immobilised on a substrate.

The esterase can be immobilised on any suitable substrate using techniques known to those skilled in the art. For example, the esterase may be immobilised on a support resin by ion exchange, adsorption (e.g. hydrophobic adsorption), or covalent coupling.

In one embodiment, the esterase is immobilised on a support resin.

In one embodiment, the esterase is immobilised on an ion exchange resin.

Those skilled in the art will be familiar with the general principle of enzymatic immobilisation technology and that principle can advantageously be applied in the context of immobilising the esterase on a substrate in accordance with the present invention.

Suitable ion-exchange resins for immobilising the esterase will generally comprise a polymer matrix or a polymer/ceramic hybrid matrix. An example of such a resin includes, but is not limited to, CM Ceramic HyperDR Ion Exchange Chromatography Resin.

In one embodiment, the ion exchange resin is a cationic exchange resin.

The method in accordance with the invention can advantageously be performed in a batch wise, semicontinuous or continuous manner.

For continuous operation of the method in accordance with the invention, the esterase will typically be immobilised on a support resin and loaded into a column. The transesterification composition produced in step (i) might then be continuously passed through the column so as to promote conversion of one or both of a mono-ester and diester terephthalate C₆-C₁₀ mono-alcohol derivative into terephthalic acid using the immobilised esterase.

Prior to being passed through the column, the transesterification composition produced in step (i) might first be diluted in a solvent such as one herein described and be combined with one or more additives for adjusting/stabilising pH and/or ion content. Such additives might include, for example, pH buffer and/or an ionic compound such as NaCl.

Step (ii) in accordance with the method of the invention produces a composition comprising the terephthalic acid.

That composition comprising the terephthalic acid may also comprise C₆-C₁₀ mono-alcohol.

If desired the so formed terephthalic acid can be readily purified/isolated from the reaction composition produced in step (ii).

Purification and/or isolation of the terephthalic acid can be performed using techniques well-known to those skilled in the art. Examples of such purification and/or isolation techniques include, but are not limited to, solvent extraction, solvent washing, filtration, distillation, solvent evaporation, column chromatography and crystallisation.

In one embodiment, the terephthalic acid produced in step (ii) is purified by acidifying of the composition comprising the terephthalic acid, for example to a pH of around 2-4 to promote precipitation of the terephthalic acid. The precipitated terephthalic acid may then be washed and collected by filtration.

C₆-C₁₀ mono-alcohol present in the composition comprising the so formed terephthalic acid may be isolated and recycled for use in the method of the invention.

### EXAMPLES

### Example 1: Base-Catalyzed Transesterification of Polyethylene Terephthalate (PET) at Varying Temperature

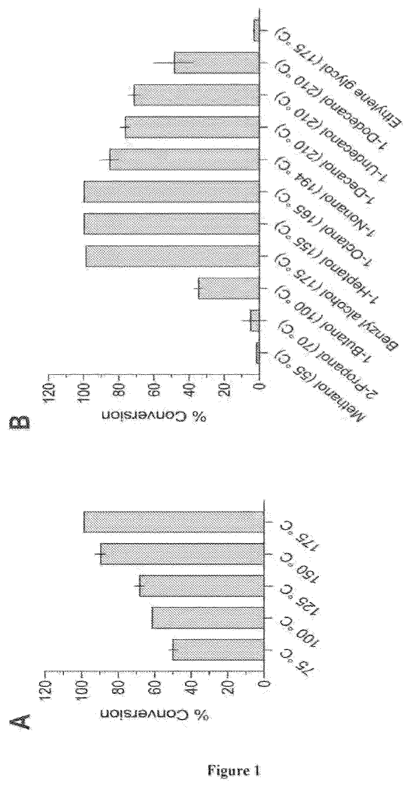

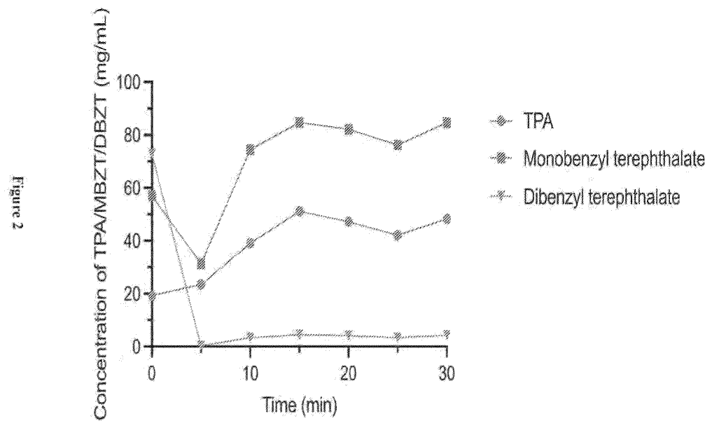

The base catalyzed transesterification reaction between PET and benzyl alcohol was performed at temperatures 75, 100, 125, 150 and 175° C. 5 mL of benzyl alcohol was added to a 25 mL RBF and heated to the respective temperature. Sodium hydroxide (70 mg) and PET (700 mg) were added successively to the hot benzyl alcohol. The reaction was stirred for 5 minutes before cooling to room temperature. Gas chromatography-mass spectrometry (GC-MS) analysis of the base-catalysed transesterification of PET with benzyl alcohol at 75° C. was performed. Benzyl alcohol elutes with a retention time of 5.086 and an abundance of approximately 1E7 and DBZT elutes with a retention time of 19.232 and an abundance of approximately 4.5E6. GC-MS analysis of the base-catalysed transesterification of PET with benzyl alcohol at 100° C. was performed. Benzyl alcohol elutes with a retention time of 5.086 and an abundance of approximately IE7 and DBZT elutes with a retention time of 19.232 and an abundance of approximately 4.5E6. GC-MS analysis of the base-catalysed transesterification of PET with benzyl alcohol at 125° C. was performed. Benzyl alcohol elutes with a retention time of 5.086 and an abundance of approximately 1E7 and DBZT elutes with a retention time of 19.232 and an abundance of approximately 6E6. GC-MS analysis of the base-catalysed transesterification of PET with benzyl alcohol at 150° C. was performed. Benzyl alcohol elutes with a retention time of 5.086 and an abundance of approximately 1E7 and DBZT elutes with a retention time of 19.232 and an abundance of approximately 7E6. GC-MS analysis of the base-catalysed transesterification of PET with benzyl alcohol at 175° C. was performed. Benzyl alcohol elutes with a retention time of 5.086 and an abundance of approximately 1E7 and DBZT elutes with a retention time of 19.232 and an abundance of approximately 9E6. All spectroscopic data are in agreement with literature values and confirm the presence of DBZT. These results are shown in FIG. 1.

To determine the dry mass of remaining PET, the reaction mixture was washed with ethyl acetate and decanted to separate the solvent and organic material from unreacted PET. The remaining PET was then dried under vacuum to remove solvent before weighing the dry mass.

### Example 2: Base Catalyzed Transesterification of PET in Varying Solvents

The base catalysed transesterification reaction between PET and various alcohols were performed as in Example 1, except the reaction temperature was chosen to be just below the boiling temperature of each solvent under investigation. Determination of the dry mass of remaining PET and product characterization was performed as in Example 1. These results are shown in FIG. 1.

GC-MS analysis of base-catalysed transesterification of PET with butanol at 100° C. confirmed the presence of dibutyl terephthalate (ESI M−H m/z 221.1). Reaction conversion=35%.

GC-MS analysis of base-catalysed transesterification of PET with 1-heptanol at 155° C. confirmed the presence of diheptyl terephthalate (ESI M+Na m/z 385.3) and monoheptyl terephthalate (ESI M−H m/z 263.0). Reaction conversion=100%.

GC-MS analysis of base-catalysed transesterification of PET with octanol at 165° C. confirmed the presence of dioctyl terephthalate (ESI M+Na m/z 413.2) and monooctyl terephthalate (ESI M−H m/z 277.1). Reaction conversion=100%

GC-MS analysis of base-catalysed transesterification of PET with 1-nonanol at 195° C. confirmed the presence of dinonyl terephthalate (ESI M+Na m/z 441.3) and monononyl terephthalate (ESI M−H m/z 291.3). Reaction conversion=85%.

GC-MS analysis of base-catalysed transesterification of PET with I-decanol at 210° C. confirmed the presence of didecyl terephthalate (ESI M+H m/z 447.3) and monodecyl terephthalate (ESI M−H m/z 305.2). Reaction conversion=77%.

GC-MS analysis of base-catalysed transesterification of PET with 1-undecanol at 210° C. confirmed the presence of diundecyl terephthalate (ESI M+H m/z 475.1). Reaction conversion=72%.

GC-MS analysis of base-catalysed transesterification of PET with I-dodecanol at 210° C. confirmed the presence of didodecyl terephthalate (ESI M+H m/z 503.3) and monodedecyl terephthalate (ESI M−H m/z 333.1). Reaction conversion=49%.

### Example 3: Purification of Terephthalate Esters by Column Chromatography

The transesterification of post-consumer PET was performed at 175° C., scaling up the reaction conditions specified in Example 1. 100 mL of 1-octanol or benzyl alcohol was added to a RB flask and heated to 175° C. 1.4 g of NaOH, and 14 g of postconsumer PET were added sequentially. The reaction was stirred for 10 minutes before cooling to room temperature and stirred with 20 mL of ethyl acetate. The heterogeneous reaction mixture was filtered through a plug of celite to get a mixture of alcohol solvent, dialkyl terephthalate and monoalkyl terephthalate esters (wherein alkyl comprises benzyl or 1-octyl groups). The filtrate was concentrated under reduced pressure to remove ethyl acetate and other volatiles. This mixture of products were further purified by flash column chromatography (silica gel, 30% ethyl acetate in hexane).

Flash column chromatography purification (silica gel, 3:7 to 9:1, ethyl acetate/hexane) of the crude reaction mixture resulting from the base-catalyzed transesterification of PET in benzyl alcohol afforded dibenzyl terephthalate and monobenzyl terephthalate as white solids. The isolated products were characterized by 1H NMR and 13C NMR and the analytical data matched literature values. Dibenzyl terephthalate: 1H NMR (400 MHZ, CDCl3) δ 8.13 (s, 4H), 7.53-7.29 (m, 10H), 5.39 (s, 4H) ppm: 13C NMR (100 MHz, CDCl3) δ 165.7, 135.8, 134.1, 129.8, 128.8, 128.6, 128.4, 67.3 ppm. Monobenzyl terephthalate: 1H NMR (400 MHZ, d6-DMSO) § 8.36-7.90 (m, 4H), 7.67-7.16 (m, 5H), 5.39 (s, 2H) ppm: 13C NMR (100 MHz, d6-DMSO) δ 166.6, 165.0, 135.9, 134.9, 133.1, 129.7, 129.5, 128.6, 128.2, 128.1, 66.6 ppm.

Flash column chromatography purification (silica gel, 1:9 to 1:0, ethyl acetate/hexane) of the crude reaction mixture resulting from the base-catalyzed transesterification of PET in 1-octanol afforded dioctyl terephthalate and monooctyl terephthalate as white solids. The isolated products were characterized by ¹H NMR and ¹³C NMR spectroscopy and the analytical data matched literature values. Dioctyl terephthalate: 1H NMR (400 MHz, d6-DMSO) δ 8.08 (s, 4H), 4.30 (t, J=6.6 Hz, 4H), 1.71 (dt, J=8.1, 6.5 Hz, 4H), 1.48-1.20 (m, 20H), 0.89-0.81 (m, 6H) ppm. 13C NMR (100 MHz, CDCl3) δ 166.1, 134.4, 129.6, 65.7, 31.9, 29.4, 29.3, 28.8, 26.2, 22.8, 14.2 ppm.

Mono-octyl terephthalate: 1H NMR (400 MHZ, d6-DMSO) δ 8.05 (m, 4H), 4.29 (t, J=6.5 Hz, 2H), 1.75 (m, 2H), 1.28 (m, 10H), 0.84 (m, 3H) ppm.

13C NMR (100 MHz, d6-DMSO) 167.1, 165.6, 135.3, 133.9, 130.1, 129.7, 65.6, 31.7, 29.1, 29.0, 28.6, 25.9, 22.5, 14.4 ppm.

### Example 4: Base Hydrolysis of Dibenzyl Terephthalate to Produce a Mixture of Monobenzyl Terephthalate and Terephthalic Acid

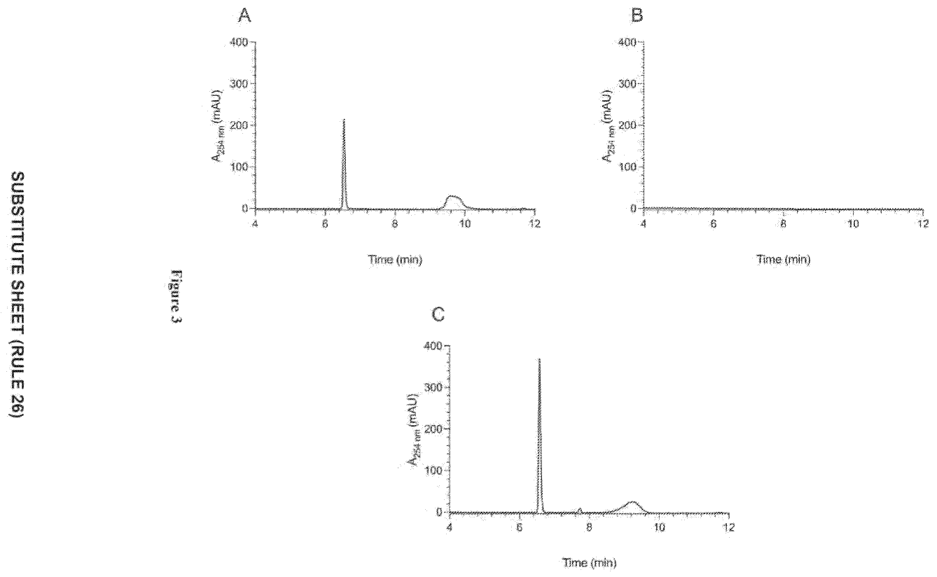

1000 mL of the crude product from the transesterification of PET by benzyl alcohol was heated to 60° C. 200 mL of 18.5 w/v % sodium hydroxide solution was added and stirred at 600 rpm for 30 minutes. Samples of the reaction mixture were taken for analysis by HPLC every 5 minutes. Concentrations of TPA, monobenzyl terephthalate and dibenzyl terephthalate in the samples were determined by comparison to a calibration curve of standards. The data are shown in FIG. 2.

### Example 5: Enzymatic Hydrolysis of Dibenzyl Terephthalate

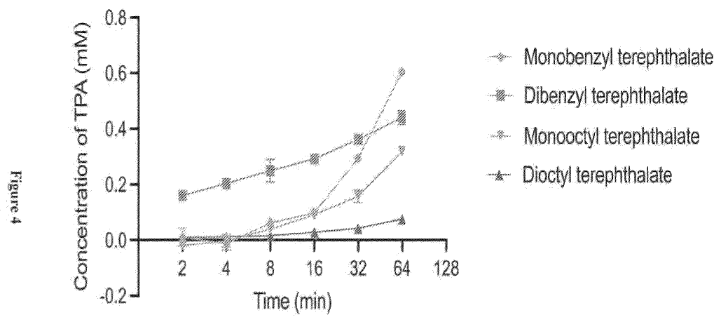

A 100 mM stock of dibenzyl terephthalate was prepared in 100% DMSO. Using this stock, a 1 mL solution was prepared containing 3.75 mM dibenzyl terephthalate (3.75% DMSO), 1 μM of an engineered esterase (SEQ ID NO:4) and 50 mM Bicine pH 9. A 1 mL control solution containing 3.75 mM dibenzyl terephthalate in 50 mM Bicine pH 9 was also prepared. The solutions were incubated at 60° C. for 1 hour and then quenched on ice. The samples were subsequently analysed using high-performance liquid chromatography (HPLC). Terephthalic acid (TPA) production was determined by comparison to TPA standards (Sigma Aldrich, >98% purity). Analytical results are shown in FIG. 3.

### Example 6: Enzymatic Hydrolysis of Monobenzyl Terephthalate, Dibenzyl Terephthalate, Monooctyl Terephthalate or Dioctyl Terephthalate

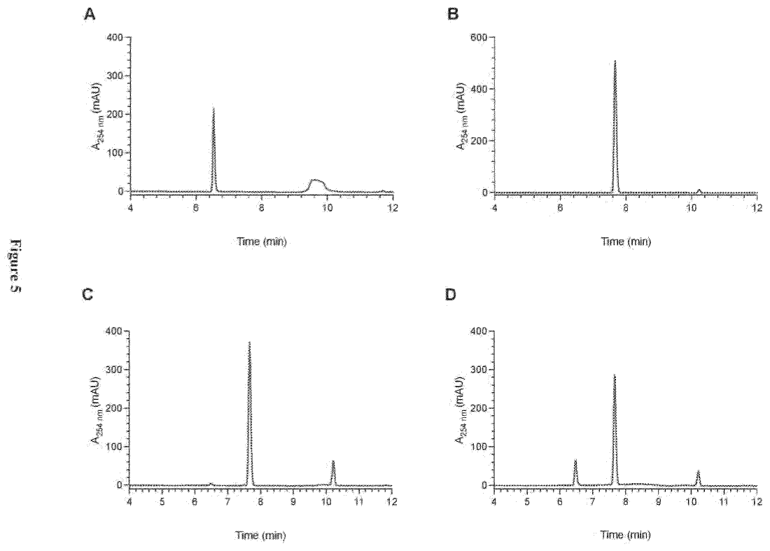

Assays of enzymatic activity against monobenzyl terephthalate, dibenzyl terephthalate, monooctyl terephthalate or dioctyl terephthalate were conducted with 1.5 mM substrate, 5% DMSO, 200 nM of an engineered esterase (SEQ ID NO: 29), and 45 mM NaH₂PO₄, 90 mM NaCl, pH 7.5. Reactions were incubated at 50° C. for 1 hour and quenched at varying time points by heating at 95° C. for at least 10 minutes. The reactions were analysed using high-performance liquid chromatography (HPLC) and compared to control reactions containing no enzyme. The concentration of terephthalic acid (TPA) was determined by comparison to a calibration curve of TPA standards (Sigma Aldrich, >98% purity). The data is shown in FIG. 4, which demonstrates the engineered esterase corresponding to SEQ ID NO. 29 catalyses hydrolysis of monobenzyl terephthalate, dibenzyl terephthalate, monooctyl terephthalate or dioctyl terephthalate into TPA.

### Example 7: Base Catalyzed Transesterification of PET in Mixture of Benzyl Alcohol and Ethylene Glycol

The base catalyzed transesterification reaction between PET and benzyl alcohol was performed in 1:4 volume ratio of benzyl alcohol:ethylene glycol. 5 mL of 1:4 benzyl alcohol:ethylene glycol was added to a 25 mL RBF and heated to 175° C. Sodium hydroxide (70 mg) and PET (700 mg) were added successively to the hot solvent mixture. The reaction was stirred for 2 hours, or until pieces of PET were no longer visible in the reaction. GC-MS analysis of the product confirmed the presence of 2-hydroxyethyl benzyl terephthalate, eluting with a retention time of 16 minutes. The spectroscopic data were in agreement with standards for 2-hydroxyethyl benzyl terephthalate in the National Institute of Standards and Technology (NIST) Mass Spectral Library.

### Example 8: Hydrolysis of Monobenzyl Terephthalate by a PETase

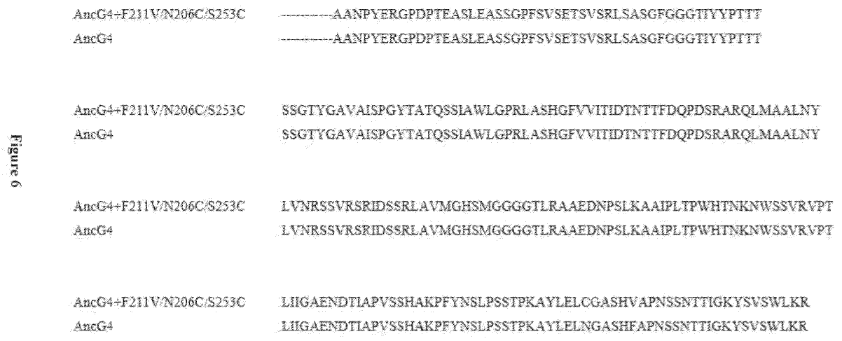

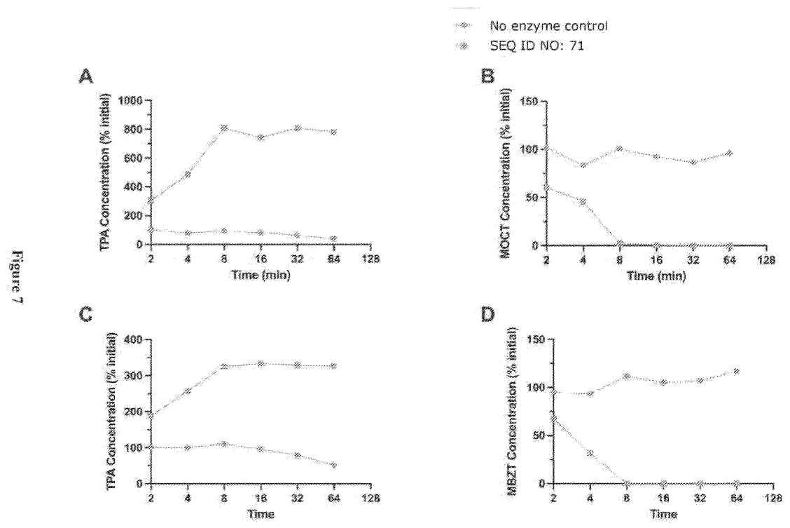

3 mg of an engineered monobenzyl terephthalate hydrolase (SEQ ID NO:29) was immobilized on 7 mL of CM Ceramic HyperD® Ion Exchange Chromatography Resin (loaded in 20 mM HEPES pH 8.0, 50 mM NaCl) pre-packed in a XK 16/20 column (Cytiva). A mixture of monobenzyl terephthalate and terephthalic acid in benzyl alcohol (produced as in Example 5) was diluted to 2% v/v in 20 mM Bicine, 100 mM NaCl pH 9 buffer. A sample of this solution was taken for HPLC analysis as a control. The solution was then passed over the column containing immobilized enzyme at a flow rate of 3 mL/min and the flow-through was collected for HPLC analysis and compared to the control. Analytical results are shown in FIG. 5. The engineered monobenzyl terephthalate hydrolase of SEQ ID NO:29 is a variant of the hydrolase of SEQ ID NO:4 that has been modified with amino acid substitutions F210V, N205C and S252C (amino acid positions corresponding to SEQ ID NO:29). The modifications imparted improved activity for mono- and di-terephthalic esters and improved thermostability. An alignment of SEQ ID NO:4 and SEQ ID NO:29 is shown in FIG. 6.

### Example 9: Hydrolysis of Monoester Terephthalates by MHETase Enzymes

The native substrate of MHETase is mono-(2-hydroxyethyl) terephthalate (MHET), a monoester of terephthalic acid (TPA). However, the inventors surprisingly found that certain enzymes having MHETase activity were also able to hydrolyse other monoesters of TPA formed by the base catalyzed transesterification reaction between PET and with a C₆-C₁₀ mono-alcohol, into TPA. For example, it was found that MHETase enzymes, such as the MHETase of SEQ ID NO: 71 could hydrolyse monobenzyl terephthalate and monooctyl terephthalate into TPA (FIG. 7A-D).

### Example 10: Hydrolysis of Monoester Terephthalates by Carboxylesterases

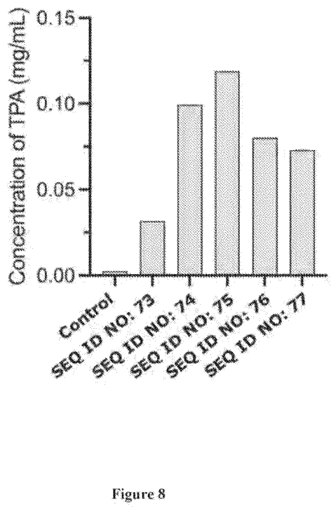

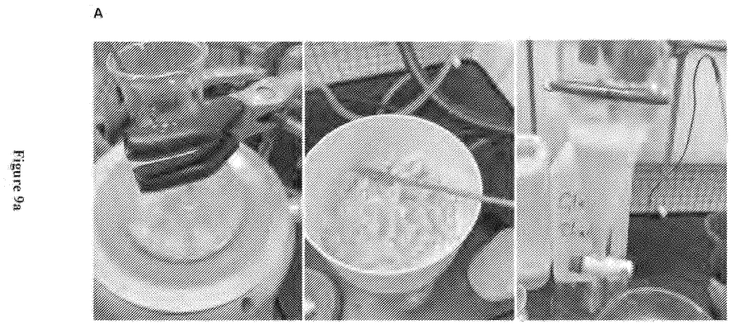

A recently discovered esterase, LAE6 from a metagenome of Lake Arreo (Martinez-Martinez et al. (2013) Appl Environ Microbiol. 79:3553) was discovered to be capable of hydrolysing monoesters of TPA formed by the base catalyzed transesterification reaction between PET and with a C₆-C₁₀ mono-alcohol, into TPA. LAE6 and its homologues were shown to hydrolyse monooctyl terephthalate into TPA (FIG. 8 and Table 2).

TABLE 2
TPA produced by LAE6 and its homologues after incubation
with 1.5 mM MOCT at room temperature (23° C.) for 1 hour

| | | Concentration of TPA |
|---|---|---|
| | | produced in reaction with |
| Variant | SEQ ID NO: | MOCT (mg/mL) |
| LAE6 | 73 | 0.0318 |
| MBI2735083.1 (C11) | 74 | 0.0994 |
| MBL0899975.1 (D11) | 75 | 0.1189 |
| TAJ91161.1 (F11) | 76 | 0.0803 |
| MBS0519452.1 (H11) | 77 | 0.0731 |

### Example 11: Extraction and Recycling of PET from Mixed Materials

2 g of material comprising 53% nylon, 44% polyester, 3% elastane was contacted with 100 mg NaOH in 35 mL benzyl alcohol at 175° C. The material was fully dissolved and formation of benzyl esters from polyester was observed. The solution was cooled, and solidified as a colourless gel. 30 mL Water was added, and a significant amount of white precipitated nylon was formed and filtered. It was observed that benzyl esters were present in the filtrate, and the collected solid contained only nylon and benzyl alcohol. The benzyl esters can be extracted and hydrolysed using esterases, in the methods disclosed herein, including one or more of the esterases described herein.

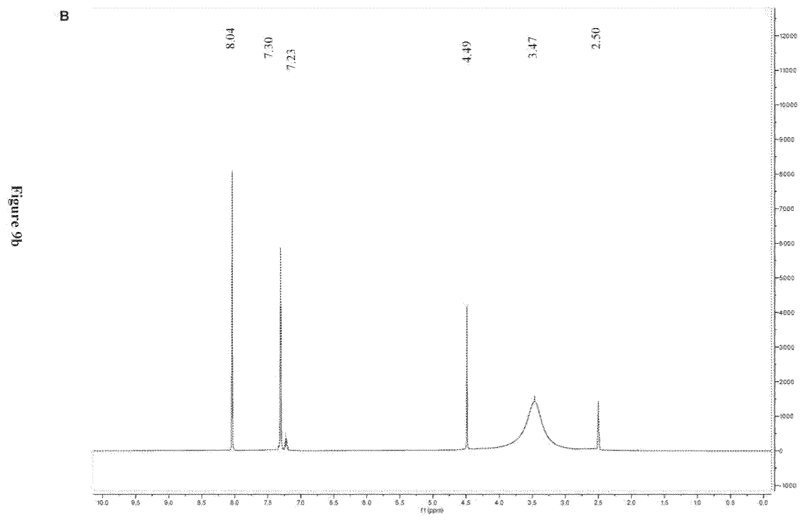

As a proof of principle experiment, and to quantify the amount of reacted polyester, the filtrate was transferred to another reaction flask and fully hydrolysed with NaOH. This solution was transferred to a separatory funnel where the alcohol and water layers clearly separated, showing the white dye in the fabric was isolated in the alcohol layer, and the water layer contained only terephthalic acid. (FIG. 9A-B).

Experiments were also conducted on drink packaging plastic bags that are approximately 12% polyester, 88% polyethylene. 18 g of material was roughly chopped up to 2×2 cm pieces, and contacted with 250 mL benzyl alcohol heated to 175° C. 600 mg of sodium hydroxide was added, and left to stir overnight. After the overnight stirring, and as proof of concept principle to detect PET, 2 mL of 6 M NaOH was added along with 8 mL water, and left to stir at 100° C. for 1 hour. 250 ml of water was added, and allowed to separate. The water layer remained opaque. The water layer was collected and TPA precipitated with sulfuric acid, and collected via vacuum filtration. 1H NMR spectroscopy showed presence of TPA.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

1. A method for recycling polyethylene terephthalate (PET), the method comprising:
(i) subjecting the PET to base-catalysed transesterification with a C₆-C₁₀ mono-alcohol to produce a composition comprising one or both of a mono-ester and di-ester terephthalate C₆-C₁₀ mono-alcohol derivatives; and
(ii) converting the so formed one or both of mono-ester and di-ester terephthalate C₆-C₁₀ mono-alcohol derivatives into terephthalic acid using an esterase.

2. The method according to claim 1, wherein the base-catalysed transesterification is performed at a temperature ranging from about 100° C. to about 180° C.

3. The method according to claim 2, wherein the PET and C₆-C₁₀ mono-alcohol are combined and heated to a temperature from about 100° C. to about 180° C.

4. The method according to claim 2, wherein the C₆-C₁₀ mono-alcohol is heated to a temperature from about 100° C. to about 180° C. and then combined with the PET.

5. The method according to claim 1, wherein the transesterification is catalysed using an alkali metal base.

6. The method according to claim 5, wherein the alkali metal base is sodium hydroxide.

7. The method according to claim 1, wherein the C₆-C₁₀ mono-alcohol is selected from hexanol, heptanol, octanol, nonanol, decanol and benzyl alcohol.

8. The method according to claim 1, wherein prior to undertaking step (ii) the composition produced in step (i) is processed to increase purity of the so formed one or both of mono-ester and di-ester terephthalate C₆-C₁₀ mono-alcohol derivatives, with the resulting processed composition being used in step (ii).

9. The method according to claim 1, wherein prior to undertaking step (ii) the composition produced in step (i) is processed to convert some or all of any di-ester terephthalate C₆-C₁₀ mono-alcohol derivative present into mono-ester terephthalate C₆-C₁₀ mono-alcohol derivative, with the resulting processed composition being used in step (ii).

10. The method according to claim 1, wherein esterase is a polyesterase, diesterase or monoesterase.

11. The method according to claim 1, wherein step (ii) produces a composition comprising terephthalic acid that is subjected to one or more techniques to increase the purity of the so formed terephthalic acid.

12. The method according to claim 1, wherein the esterase is immobilised on a substrate.

13. The method according to claim 12, wherein the substrate is a support resin.

14. The method according to claim 1 that is performed in a continuous mode.

15. The method according to claim 1, wherein the PET that is to be recycled is provided in the form of a feedstock material comprising one or more co-materials that do not undergo reaction with the C₆-C₁₀ mono-alcohol.

16. The method according to claim 15, wherein after step (i) the one or more co-materials are isolated from the composition produced in step (i).

17. The method according to claim 1, wherein the esterase comprises an amino acid sequence selected from the group consisting of SEQ ID NOs: 2-29 and an amino acid sequence having at least 70% sequence identity thereto.

18. The method according to claim 17, wherein the esterase comprises an amino acid sequence of SEQ ID NO:29 or an amino acid sequence having at least 70% sequence identity thereto.

19. A polypeptide having esterase activity, wherein the polypeptide comprises an amino acid sequence of SEQ ID NO:29 or an amino acid sequence having at least 70% sequence identity thereto.

20. The method according to claim 1, wherein the esterase is
a) a PETase, wherein the PETase comprises an amino acid sequence of SEQ ID NO: 1 or an amino acid sequence having at least 70% sequence identity thereto;
b) a carboxylesterase, wherein the carboxylesterase comprises an amino acid sequence of SEQ ID NO: 73, or an amino acid sequence that has at least 70% sequence identity to SEQ ID NO: 73; and/or
c) a MHETase, wherein the MHETase comprises an amino acid that
(i) has at least 70% sequence identity to SEQ ID NO: 31, and
(ii) differs from SEQ ID NO:31 by an amino acid substitution at one or more positions selected from the group consisting of positions that correspond to amino acid positions 156 to 396, 398 to 410 and 425 to 603 of SEQ ID NO: 31.

21. The method according to claim 20, wherein the esterase is:
a) a PETase comprising the amino acid sequence of SEQ ID NO:29;
b) a carboxylesterase comprising the amino acid sequence of SEQ ID NO: 73-77; and/or
a MHETase comprising the amino acid sequence of SEQ ID NO:71.


SEQUENCE LISTING (79 sequences)

SEQ ID NO: 1 - protein, 290 aa, synthetic construct
MNFPRASRLMQAAVLGGLMAVSAAATAQTNPYARGPNPTAASLEASAGPFTVRSFTVSRPSGYGAGTVYYPTNAGGTVGAIAIVPGYTARQSSIKWWGPRLASHGFVVITIDTNSTLDQPSSRSSQQMAALRQVASLNGTSSSPIYGKVDTARMGVMGWSMGGGGSLISAANNPSLKAAAPQAPWDSSTNFSSVTVPTLIFACENDSIAPVNSSALPIYDSMSRNAKQFLEINGGSHSCANSGNSNQALIGKKGVAWMKRFMDNDTRYSTFACENPNSTRVSDFRTANCS

SEQ ID NO: 2 - protein, 257 aa, synthetic construct
MYERGPDPTEASLEASSGPFSVSETSVSRLSASGFGGGTIYYPTTTSSGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSVSWLKRFVDNDTRYSQFLCPAPHDDSAISEYRSTCPY

SEQ ID NO: 3 - protein, 257 aa, synthetic construct
MYERGPDPTESSLEASSGPFSVSETSVSRLSASGFGGGTIYYPTTTSEGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSIAWLKRFVDNDTRYSQFLCPAPHDDSAISEYRSTCPY

SEQ ID NO: 4 - protein, 260 aa, synthetic construct
AANPYERGPDPTEASLEASSGPFSVSETSVSRLSASGFGGGTIYYPTTTSSGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSVSWLKRFVDNDTRYSQFLCPAPHDDSAISEYRSTCPY

SEQ ID NO: 5 - protein, 260 aa, synthetic construct
AANPYERGPDPTESSLEASSGPFSVSETSVSRLSASGFGGGTIYYPTTTSEGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSIAWLKRFVDNDTRYSQFLCPAPHDDSAISEYRSTCPY

SEQ ID NO: 6 - protein, 260 aa, synthetic construct
ADNPYERGPDPTEASLEASSGPFSVSETSVSRLSASGFGGGTIYYPTTTSSGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSVSWLKRFVDNDTRYSQFLCPAPHDDSAISEYRSTCPY

SEQ ID NO: 7 - protein, 260 aa, synthetic construct
AANPYERGPEPTEASLEASSGPFSVSETSVSRLSASGFGGGTIYYPTTTSSGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSVSWLKRFVDNDTRYSQFLCPAPHDDSAISEYRSTCPY

SEQ ID NO: 8 - protein, 260 aa, synthetic construct
AANPYERGPDPTEASVEASSGPFSVSETSVSRLSASGFGGGTIYYPTTTSSGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSVSWLKRFVDNDTRYSQFLCPAPHDDSAISEYRSTCPY

SEQ ID NO: 9 - protein, 260 aa, synthetic construct
AANPYERGPDPTEASLEASSGPFSVSETSVSRLSASGFGGGTIYYPTTTSAGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSVSWLKRFVDNDTRYSQFLCPAPHDDSAISEYRSTCPY

SEQ ID NO: 10 - protein, 260 aa, synthetic construct
AANPYERGPDPTEASLEASSGPFSVSETSVSRLSASGFGGGTIYYPTTTSSGTYGAVAISPGYTATQSSIAWLGPRLASHGFVVITIDTQTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSVSWLKRFVDNDTRYSQFLCPAPHDDSAISEYRSTCPY

SEQ ID NO: 11 - protein, 260 aa, synthetic construct
AANPYERGPDPTEASLEASSGPFSVSETSVSRLSASGFGGGTIYYPTTTSSGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKDWSSVRVPTLIIGAENDTIAPVSSHAKPFYNSLPSSTPKAYLELNGASHFAPNSSNTTIGKYSVSWLKRFVDNDTRYSQFLCPAPHDDSAISEYRSTCPY

SEQ ID NO: 12 - protein, 260 aa, synthetic construct
AANPYERGPDPTEASLEASSGPFSVSETSVSRLSASGFGGGTIYYPTTTSSGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPASTPKAYLELNGASHFAPNSSNTTIGKYSVSWLKRFVDNDTRYSQFLCPAPHDDSAISEYRSTCPY

SEQ ID NO: 13 - protein, 260 aa, synthetic construct
AANPYERGPDPTEASLEASSGPFSVSETSVSRLSASGFGGGTIYYPTTTSSGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSVSWLKRFVDNDTRYNQFLCPAPHDDSAISEYRSTCPY

SEQ ID NO: 14 - protein, 260 aa, synthetic construct
AANPYERGPDPTDSSLEASSGPFSVSETSVSRLSASGFGGGTIYYPTTTSEGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSIAWLKRFVDNDTRYSQFLCPAPHDDSAISEYRSTCPY

SEQ ID NO: 15 - protein, 260 aa, synthetic construct
AANPYERGPDPTESSLEASSGPFSVSQTSVSRLSASGFGGGTIYYPTTTSEGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSIAWLKRFVDNDTRYSQFLCPAPHDDSAISEYRSTCPY

SEQ ID NO: 16 - protein, 260 aa, synthetic construct
AANPYERGPDPTESSLEASSGPFSVSETSVSRLSVSGFGGGTIYYPTTTSEGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSIAWLKRFVDNDTRYSQFLCPAPHDDSAISEYRSTCPY

SEQ ID NO: 17 - protein, 260 aa, synthetic construct
AANPYERGPDPTESSLEASSGPFSVSETSVSRLSASGFGGGTIYYPTSTSEGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSIAWLKRFVDNDTRYSQFLCPAPHDDSAISEYRSTCPY

SEQ ID NO: 18 - protein, 260 aa, synthetic construct
AANPYERGPDPTESSLEASSGPFSVSETSVSRLSASGFGGGTIYYPTTTSSGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSIAWLKRFVDNDTRYSQFLCPAPHDDSAISEYRSTCPY

SEQ ID NO: 19 - protein, 260 aa, synthetic construct
AANPYERGPDPTESSLEASSGPFSVSETSVSRLSASGFGGGTIYYPTTTSEGTFGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSIAWLKRFVDNDTRYSQFLCPAPHDDSAISEYRSTCPY

SEQ ID NO: 20 - protein, 260 aa, synthetic construct
AANPYERGPDPTESSLEASSGPFSVSETSVSRLSASGFGGGTIYYPTTTSEGTYGAVAISPGYTATQSSIAWLGRRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSIAWLKRFVDNDTRYSQFLCPAPHDDSAISEYRSTCPY

SEQ ID NO: 21 - protein, 260 aa, synthetic construct
AANPYERGPDPTESSLEASSGPFSVSETSVSRLSASGFGGGTIYYPTTTSEGTYGAVAISPGYTATQSSIAWLGPRLASHGFVVITIDTNTTLDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSIAWLKRFVDNDTRYSQFLCPAPHDDSAISEYRSTCPY

SEQ ID NO: 22 - protein, 260 aa, synthetic construct
AANPYERGPDPTESSLEASSGPFSVSETSVSRLSASGFGGGTIYYPTTTSEGTYGAVAISPGYTATQSSIAWLGPRLASHGFVVITIDTNTTFDQPDSRARQLMAALDYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSIAWLKRFVDNDTRYSQFLCPAPHDDSAISEYRSTCPY

SEQ ID NO: 23 - protein, 260 aa, synthetic construct
AANPYERGPDPTESSLEASSGPFSVSETSVSRLSASGFGGGTIYYPTTTSEGTYGAVAISPGYTATQSSIAWLGPRLASHGFVVITIDTNTTFDQPDSRARQLMAALNYLVNRSSVRSRIDPSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSIAWLKRFVDNDTRYSQFLCPAPHDDSAISEYRSTCPY

SEQ ID NO: 24 - protein, 260 aa, synthetic construct
AANPYERGPDPTESSLEASSGPFSVSETSVSRLSASGFGGGTIYYPTTTSEGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNTSNTTIGKYSIAWLKRFVDNDTRYSQFLCPAPHDDSAISEYRSTCPY

SEQ ID NO: 25 - protein, 260 aa, synthetic construct
AANPYERGPDPTESSLEASSGPFSVSETSVSRLSASGFGGGTIYYPTTTSEGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSISWLKRFVDNDTRYSQFLCPAPHDDSAISEYRSTCPY

SEQ ID NO: 26 - protein, 260 aa, synthetic construct
AANPYERGPDPTESSLEASSGPFSVSETSVSRLSASGFGGGTIYYPTTTSEGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSIAWLKRFVDNDTRYTQFLCPAPHDDSAISEYRSTCPY

SEQ ID NO: 27 - protein, 260 aa, synthetic construct
AANPYERGPDPTESSLEASSGPFSVSETSVSRLSASGFGGGTIYYPTTTSEGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSIAWLKRFVDNDTRYSQFLCPAPSDDSAISEYRSTCPY

SEQ ID NO: 28 - protein, 260 aa, synthetic construct
AANPYERGPDPTESSLEASSGPFSVSETSVSRLSASGFGGGTIYYPTTTSEGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSIAWLKRFVDNDTRYSQFLCPAPHDDAAISEYRSTCPY

SEQ ID NO: 29 - protein, 260 aa, synthetic construct
AANPYERGPDPTEASLEASSGPFSVSETSVSRLSASGFGGGTIYYPTTTSSGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELCGASHVAPNSSNTTIGKYSVSWLKRFVDNDTRYSQFLCPAPHDDSAICEYRSTCPY

SEQ ID NO: 30 - protein, 4 aa, synthetic construct
AANP

SEQ ID NO: 31 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRNFATIATDGGHDNAVNDNPDALGTVAFGLDPQARLDMGYNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 32 - protein, 524 aa, synthetic construct
PATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGYNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 33 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 34 - protein, 584 aa, synthetic construct
GGGSTPLPLPQQQPPQQEPPPPPVPLASRAACEALKDGNGDMVWPNAAVVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 35 - protein, 584 aa, synthetic construct
GGGSTPLPLPQQQPPQQEPPPPPVPLASRAACEALKDGNGDMVWPNAATVVEVAAWRDPAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 36 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYAIKFRLRMPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 37 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRLPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 38 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMQGGSGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 39 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGGGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 40 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTDGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 41 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRNFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 42 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIGTDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 43 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLLMGVNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 44 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDYGVNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 45 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNAYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 46 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAAKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 47 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAGKALVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 48 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAPDKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 49 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAADKSYFIGCSNGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 50 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMASQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 51 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLAQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 52 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLVKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 53 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAAISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 54 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAGIAGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 55 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAGISGAWLTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 56 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAGISGAWTLQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 57 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAGISGAWTTQSLAPAAVGLDAQGVPLILKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 58 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLVSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 59 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLAQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 60 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVRAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 61 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWPWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 62 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSLMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 63 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDPAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 64 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADWYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 65 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTAANFACAAPP

SEQ ID NO: 66 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGVNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEASFACAAPP

SEQ ID NO: 67 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGYNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADYYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 68 - protein, 553 aa, synthetic construct
CEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDMGYNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADWYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 69 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRGFAVIATDGGHDNAVNDNPDALGTVAFGLDPQARLDYGYNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADWYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 70 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRNFAVIATDGGHDNAVNDNPDALGTVAFGLDPQARLDYGYNSYDQVTQAGKAAVARFYGRAADKSYFIGCSEGGREGMMLSQRFPSHYDGIVAGAPGYQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADWYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 71 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRNFAVIATDGGHDNAVNDNPDALGTVAFGLDPQARLDYGYNSYDQVTQAGKAAVARFYGRAADKSYFIGCSEGGREGMMLSQRFPSHYDGIVAGAPGFQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADWYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 72 - protein, 584 aa, synthetic construct
GGGSTPLPLPQQQPPQQEPPPPPVPLASRAACEALKDGNGDMVWPNAATVVEVAAWRDAAPATASAAALPEHCEVSGAIAKRTGIDGYPYEIKFRLRMPAEWNGRFFMEGGSGTNGSLSAATGSIGGGQIASALSRNFAVIATDGGHDNAVNDNPDALGTVAFGLDPQARLDMGYNSYDQVTQAGKAAVARFYGRAADKSYFIGCSEGGREGMMLSQRFPSHYDGIVAGAPGFQLPKAGISGAWTTQSLAPAAVGLDAQGVPLINKSFSDADLHLLSQAILGTCDALDGLADGIVDNYRACQAAFDPATAANPANGQALQCVGAKTADCLSPVQVTAIKRAMAGPVNSAGTPLYNRWAWDAGMSGLSGTTYNQGWRSWWLGSFNSSANNAQRVSGFSARSWLVDFATPPEPMPMTQVAARMMKFDFDIDPLKIWATSGQFTQSSMDWHGATSTDLAAFRDRGGKMILYHGMSDAAFSALDTADWYERLGAAMPGAAGFARLFLVPGMNHCSGGPGTDRFDMLTPLVAWVERGEAPDQISAWSGTPGYFGVAARTRPLCPYPQIARYKGSGDINTEANFACAAPP

SEQ ID NO: 73 - protein, 315 aa, unidentified
  source 1..315: note = isolate from a metagenome
MLLPETRNLLDLMDAATRGGRPRLETLPHAVGRKAVDKMSEDGEADPPEVAEVANGGFAGPASEIRFRRYRPLGEAAGLLPTLIYYHGGGFVIGNIETHDSTCRRLANKSRCQVISIDYRLAPEHPFPAPIDDGIAAFRHIRDNAESFGADAARLAVGGDSAGGAMAAVVCQACRDAGETGPAFQMLIYPATDSSRESASRVAFAEGYFLSKALMDWFWEAYVPEDTDLTDLRLSPLLATDFTGLPPAFVLTAGYDPLRDEGRAYADRLIEAGIKTTYVNYPGTIHGFFSLTRFLSQGLKANDEAAAVMGAHFGT

SEQ ID NO: 74 - protein, 315 aa, unidentified
  source 1..315: note = Rhodospirallales
MLDHEARVLLELMEKAVQEGQPKLHTLPYAVGRQAVDKMSEDSEADPMQVGEVADGAFAGPGGEIRFRRYRPLGAAAGPLPTLIYYHGGGFVIGNIETHDSTCRRLANKSRCQVISIDYRLSPEHPFPASTDDGIAAFRHVRDNAASFGVDATRIAVGGDSAGGAIAAVVCQAIRDAGDKQGPAFQMLIYPATDSSKQSASRVAFAEGYFLTKELMEWFWKAYVPAGTDLSDLRLSPLLAEDFRGLPPAFVLTAGYDPLRDEGRAYADRLIDAGIKTTYVNYPGTIHGFFSLTRFLSQGLKANDEAAAVMAAHFG

SEQ ID NO: 75 - protein, 314 aa, Reyranella sp.
MLDQEARTLLDLMEEANKAGRPKLETLPHALGRAAVDKMSEDGEAEPMDVAEVADGGFMGPGGEIRFRKYRPLGVNAGSLPTLIYYHGGGFVIGTIETHDSTCRRLANKSRCQVISIAYRLAPEHPFPAPTDDAVAAFRHIRDNAASFGADPGRLAVGGDSAGGALAAVVCQAMRDAQEAGPAFQMLIYPATDSSKESGSRKAFAEGYFLTKSLMDWFWKAYVPAGTDLADLRLSPLLAKSFEGLPPAFVLTAGFDPLRDEGRAYADRLIDAGVKTTYVNYPGTIHGFFSMTRFLAQGLRANDEAAAVLGAHFG

SEQ ID NO: 76 - protein, 318 aa, Reyranella sp.
MLDAEARILLDLMEEATKAGRPKLETLPHAVGRAAVDKMSEDSEADPPQVGDTVDGVFAGPGGPIKYRRYLPLGVTLALTQGNLPTLIYYHGGGFVIGTIETHDSTCRRLANKSRCQVISIDYRLSPEHPFPAPIDDGIAAFRHIRDHADTFGADPARLAVGGDSAGGAVAAVVCQAMRDAKEAGPAFQMLIYPATDSSKESGSRQQFAEGYFLTKGLMDWFWKAYVPAGTDLSDLRLSPLLAKDFTGLPPAFVLTAGHDPLRDEGRAYANRLIDAGVKTTYVNYPGTIHGFFSLTRFLQQGLKANDEAAAVMGAFFG

SEQ ID NO: 77 - protein, 314 aa, unidentified
  source 1..314: note = Proteobacteria
MLDAEARILLDLMDKAVKEGRPLLHTLPHKVGRAAVDKMSEDSEADPPAVGEVTDGAFAGPDGEIRFRHYRPFGVPSGPLPTLVYYHGGGFVIGNLETHDSTCRRLANKSRCQVIAVDYRLSPEYPFPAPIDDGVAAFRHIRDHAASFGADAKRIAVGGDSAGGAMAAVVCQTCRDAGDAPPAFQMLIYPATDSSKESRSRRELAEGYFLTKDLMDWFWSAYVPAGTDLSDIRLSPLLARDFKGLPPAFVLTAGHDPLRDEGRAYADRLIDAGVKTTYVNYPGTIHGFFSLTRFLQQGLKANDEAAAVLAAHFG

SEQ ID NO: 78 - protein, 229 aa, synthetic construct
AANPYERGPDPTEASLEASSGPFSVSETSVSRLSASGFGGGTIYYPTTTSSGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELNGASHFAPNSSNTTIGKYSVSWLKR

SEQ ID NO: 79 - protein, 229 aa, synthetic construct
AANPYERGPDPTEASLEASSGPFSVSETSVSRLSASGFGGGTIYYPTTTSSGTYGAVAISPGYTATQSSIAWLGPRLASHGFVVITIDTNTTFDQPDSRARQLMAALNYLVNRSSVRSRIDSSRLAVMGHSMGGGGTLRAAEDNPSLKAAIPLTPWHTNKNWSSVRVPTLIIGAENDTIAPVSSHAKPFYNSLPSSTPKAYLELCGASHVAPNSSNTTIGKYSVSWLKR